(19) United States
(12) Patent Application Publication
(10) Pub. No.: US 2023/0028818 A1
(43) Pub. Date: Jan. 26, 2023

(54) METHODS FOR USING EXTRACELLULAR MICROVESICLES WITH SYNCYTIOTROPHOBLAST MARKERS TO DIAGNOSE PREECLAMPSIA

(71) Applicant: The Trustees Of The University of Pennsylvania, Philadelphia, PA (US)

(72) Inventor: Prashanth VALLABHAJOSYULA, New Haven, CT (US)

(73) Assignee: The Trustees Of The University of Pennsylvania, Philadelphia, PA (US)

(21) Appl. No.: 17/937,897

(22) Filed: Oct. 4, 2022

Related U.S. Application Data
(63) Continuation of application No. PCT/US2021/025929, filed on Apr. 6, 2021.
(60) Provisional application No. 63/005,517, filed on Apr. 6, 2020.

(51) Int. Cl. G01N 33/566; G01N 21/64

(52) U.S. Cl. CPC G01N 33/566; G01N 21/6428

(57) ABSTRACT

This present disclosure relates to the use of syncytiotrophoblast-derived microvesicles for diagnosing and/or monitoring a subject with preeclampsia. Accordingly, this disclosure provides for methods for isolating, purifying, and/or detecting syncytiotrophoblast-derived microvesicles from a biological fluid of a pregnant subject. The present disclosure also provides kits for diagnosing a subject with preeclampsia, where the kit contains reagents useful for isolating, purifying, and/or identifying the syncytiotrophoblast-derived microvesicles in a biological sample and for detecting one or more biomarkers present on the surface of or within the syncytiotrophoblast-derived microvesicles.

### CROSS-REFERENCE TO RELATED APPLICATIONS

This application is a continuation of International Patent Application No. PCT/US21/25929, filed Apr. 6, 2021, which claims priority to U.S. Provisional Application No. 63/005,517, filed on Apr. 6, 2020, the content of each of which is incorporated in their entirety, and to each of which priority is claimed.

### INTRODUCTION

The present disclosure relates to syncytiotrophoblast-derived microvesicles and their use in diagnosing and/or monitoring a subject with preeclampsia.

### BACKGROUND

Preeclampsia is a common pregnancy-associated disorder and affects up to 3 to 5% of pregnancies (Magee et al., 2014, Tranquilli et al., 2013, Tranquilli et al., 2014, Wallis et al., 2008). In addition to increased morbidity and mortality seen in the mother, preeclampsia is also associated with fetal complications, including premature birth and fetal growth restriction. Preeclampsia manifests as new onset hypertension (systolic blood pressure>140 mmHg and diastolic pressure>90 mmHg; proteinuria), which can occur at an early stage or late stage during pregnancy. Its severity can also vary and is classified as mild, moderate, severe preeclampsia and eclampsia (2000, Bulletins—Obstetrics, 2002, Lindheimer et al., 2010). The precise mechanistic cause of preeclampsia is under investigation, but there is wide agreement that the disorder originates in the placenta (Brosens et al., 1972, Fukui et al., 2012, George and Granger, 2010, LaMarca et al., 2008, Nelissen et al., 2011, Pijnenborg et al., 2008, Roberts and Gammill, 2005), and is related to decreased uteroplacental blood flow (Brosens et al., 1972, Damsky and Fisher, 1998, Fukui et al., 2012, LaMarca et al., 2008, Nelissen et al., 2011, Pijnenborg et al., 2008, Roberts and Gammill, 2005), which leads to a cascade of physiologic processes manifesting clinically as a pregnancy-associated hypertensive disorder (Mutter and Karumanchi, 2008, Thadhani et al., 2011, Wang et al., 2009).

The diagnosis of this condition can be based on clinical factors and typically occurs during the second trimester of pregnancy, but the mechanisms underlying the pathophysiology of preeclampsia may be initiated early during the first trimester. Early diagnosis of preeclampsia in at-risk subjects would permit closer monitoring and treatment of this condition and may allow for earlier intervention to help reduce the associated risks to the fetus and the mother. Therefore, there is a need for non-invasive and accurate methods for early diagnosis of preeclampsia.

### SUMMARY

The present disclosure provides techniques for diagnosing a subject with preeclampsia. An example method includes detecting and/or isolating one or more biomarkers from a biological sample, and diagnosing preeclampsia in the subject when there is a change in the presence and/or level of the one or more biomarkers. Another example method includes isolating, purifying, and/or identifying one or more syncytiotrophoblast-derived microvesicles from a biological sample, analyzing one or more biomarkers associated with the syncytiotrophoblast-derived microvesicles, and diagnosing the subject with preeclampsia when there is a change in the presence and/or level of the biomarkers as compared to a reference control level.

In certain embodiments, the biomarker is a protein and/or a nucleic acid. In certain embodiments, the biomarker is syncytin-1 and wherein the reduction in the level of syncytin-1 compared to the reference control is indicative that the subject has preeclampsia.

A further example method in accordance with the disclosed subject matter includes isolating, purifying, and/or identifying one or more syncytiotrophoblast-derived microvesicles from the biological sample, analyzing the number of syncytiotrophoblast-derived microvesicles expressing syncytin-1, and diagnosing the subject with preeclampsia when the number of syncytin-1-expressing syncytiotrophoblast-derived microvesicles is reduced compared to a reference control.

In certain embodiments, the method further includes detecting a marker specific for syncytiotrophoblasts and/or microvesicles. In certain embodiments, the marker specific for syncytiotrophoblasts is a protein. In certain embodiments, the protein is a surface protein. In certain embodiments, the marker specific for syncytiotrophoblasts is selected from syncytin-1, PLAP, and a combination thereof. In certain embodiments, the marker specific for microvesicles is selected from flotillin-1, CD63, CD9, CD81, TSG101, and a combination thereof.

The present disclosure also provides methods for treating a subject with preeclampsia. An example method includes isolating, purifying, and/or identifying one or more syncytiotrophoblast-derived microvesicles from a biological sample, analyzing one or more biomarkers associated with the syncytiotrophoblast-derived microvesicles, diagnosing the subject with preeclampsia when there is a change in the presence and/or level of the one or more biomarkers as compared to a reference control level;, and treating the subject diagnosed with preeclampsia. In certain embodiments, the subject is treated with one or more of administration of an anti-hypertensive medication, delivery, administration of a corticosteroid, bed rest; and/or , administration of an anti-convulsant medication.

In certain embodiments, the reference control is the level in a pregnant subject that does not have preeclampsia.

The present disclosure further provides methods for preparing a syncytiotrophoblast-derived microvesicles fraction from a pregnant subject An example method includes extracting one or more microvesicles from a biological sample of a pregnant subject, producing a fraction of the microvesicles by selectively enriching syncytiotrophoblast-derived microvesicles, and measuring one or more biomarkers.

In certain embodiments, the enriching comprises contacting the sample with an agent binding a cell-specific marker. In certain embodiments, the cell-specific marker is selected from syncytin-1, placental alkaline phosphatase (PLAP), plac-1, flotillin-1, CD63, CD9, CD81, and TSG101. In certain embodiments, the cell-specific marker is syncytin-1. In certain embodiments, the method further comprises size discrimination of microvesicles.

The present disclosure also provides methods for isolating and/or purifying syncytiotrophoblast-derived microvesicles. An example method includes obtaining a biological sample from a subject, isolating and/or purifying one or more microvesicles from the biological sample, and isolating, purifying, and/or identifying one or more syncytiotrophoblast-derived microvesicles from the one or more microvesicles by detecting a marker specific for syncytiotrophoblasts.

In certain embodiments, the marker specific for syncytiotrophoblasts is a protein. In certain embodiments, the protein is a surface protein. In certain embodiments, the marker specific for syncytiotrophoblasts is selected from the group consisting of syncytin-1, PLAP, and a combination thereof. In certain embodiments, isolating and/or purifying one or more microvesicles from the biological sample comprises detecting a marker specific for microvesicles. In certain embodiments, the marker specific for microvesicles is selected from the group consisting of flotillin-1, CD63, CD9, CD81, TSG101, and a combination thereof. In certain embodiments, the subject is human. In certain embodiments, the biological sample is a blood sample.

The present disclosure provides a kit for diagnosing and/or monitoring a subject with preeclampsia. An example kit includes reagents useful for detecting a marker specific to a syncytiotrophoblast-derived microvesicle. In certain embodiments, the kit includes a packaged probe and primer set, arrays/microarrays, marker-specific antibodies or marker-specific antibody-conjugated beads or quantum dots. In certain embodiments, the kit includes a pair of oligonucleotide primers, suitable for polymerase chain reaction or nucleic acid sequencing, for detecting the marker. In certain embodiments, the kit includes a monoclonal antibody or antigen-binding fragment thereof, or a polyclonal antibody or antigen-binding fragment thereof, for detecting the marker. In certain embodiments, the marker specific to a syncytiotrophoblast-derived microvesicle is selected from syncytin-1, PLAP, and a combination thereof.

### BRIEF DESCRIPTION OF THE DRAWINGS

FIGS. 1A-1C illustrate extracellular vesicles (EVs) released by human choriocarcinoma-derived cells (BeWo cells) express placenta-specific protein markers. FIG. 1A shows Western blot analysis of EVs released into culture supernatant from BeWo cells. FIG. 1B shows nanoparticle scatter analysis of BeWo EV preparations showed similar size distribution, with majority of EVs in the size range consistent exosomes (50-200 nm) along with lower concentrations of microvesicles of larger sizes. FIG. 1C shows NanoSight nanoparticle detector analysis for surface expression of placental proteins is shown.

FIGS. 2A-2C illustrate EVs isolated from maternal circulation express canonical exosome markers. FIG. 2A shows EVs isolated utilizing methodology detailed yielded nanoparticles enriched in exosome markers CD63, TSG101, and flotillin-1 without contamination from cellular debris and apoptotic bodies (cytochrome C). FIG. 2B shows nanoparticle scatter analysis confirmed that majority of isolated EVs had size distribution consistent with exosomes (50-200 nm), and there was no difference in the size distribution of EVs between the Preeclampsia versus Control groups (p=0.415). FIG. 2C shows total plasma EV quantities in Preeclampsia versus Control groups were also similar (p=0.313).

FIGS. 3A-3C illustrate Syncytin-1 expression in preeclampsia patients compared to healthy pregnant controls. FIG. 3A shows EVs from women were analyzed for PLAP expression by Western Blot. FIG. 3B shows maternal plasma EV samples were analyzed for syncytiotrophoblast protein marker expression by Western blot. FIG. 3C shows RT-PCR analysis of plasma EV mRNA showed similar levels of syncytin-1 in both preeclampsia and control subjects.

FIGS. 4A-4C illustrate decreased syncytin-1 EV signal in maternal circulation enables diagnosis of preeclampsia with high accuracy. FIG. 4A shows syncytiotrophoblast EVs were quantified on the nanoparticle detector using anti-syncytin-1 antibody-conjugated quantum dots.

FIG. 4B shows scatter plot analysis of STEV signal showed significantly decreased syncytin-1 signal in preeclampsia subjects (p=2.82×10⁻¹¹). FIG. 4C shows receiver operating characteristic curve (ROC) in a binary cohort of preeclampsia versus control subjects was constructed for syncytin-1 EV signal, total plasma EVs quantity, and mean plasma EV size.

### DETAILED DESCRIPTION

The present disclosure provides non-invasive methods related to the use of microvesicles, e.g., syncytiotrophoblast-derived microvesicles, to diagnose and/or monitor a subject with preeclampsia. The present disclosure provides for methods and kits for determining the presence of one or more biomarkers for preeclampsia in a biological sample of a subject, and methods for using the presence of such biomarkers to predict, diagnose, and/or monitor preeclampsia in a subject. For example, but not by way of limitation, the present disclosure provides methods for early diagnosis of preeclampsia.

The present disclosure further provides methods for isolating, purifying, and/or identifying syncytiotrophoblast-derived microvesicles released into the bodily fluids of a subject. For example, but not by way of limitation, the present disclosure provides for methods and kits for isolating, purifying, and/or identifying one or more syncytiotrophoblast-derived microvesicles in a biological sample of a subject.

### 1. Definitions

As used herein, the term “biomarker” refers to a marker (e.g., microvesicle pool profile, an expressed gene, including mRNA, and/or protein) that allows detection of a disease and/or disorder in an individual, including detection of disease in its early stages. Early stage of a disease, as used herein, refers to the time period between the onset of the disease and the time point that signs or symptoms of the disease emerge. Biomarkers, as used herein, include microvesicles (e.g., exosomes), nucleic acid, and/or protein markers or combinations thereof. In certain non-limiting embodiments, the expression level of a biomarker as determined by mRNA and/or protein levels in a biological sample from an individual to be tested is compared with respective levels in a biological sample from the same individual or another healthy individual. In certain non-limiting embodiments, the presence or absence of a biomarker as determined by mRNA and/or protein levels in a biological sample from an individual to be tested is compared with the respective presence or absence in a biological sample from the same individual, or another healthy individual. In certain non-limiting embodiments, the presence or absence of a biomarker in a biological sample of a subject is compared to a reference control.

The terms “reference sample,” “reference control” or “reference,” as used interchangeably herein, refers to a control for a biomarker that is to be detected in a biological sample of a subject. For example, but not by way of limitation, a reference can be the level of a biomarker from a healthy individual free from preeclampsia, e.g., a pregnant subject that does not have preeclampsia. In certain embodiments, a reference can be the level of a biomarker from a healthy individual that underwent treatment for preeclampsia, wherein the healthy individual is non-symptomatic. In certain embodiments, a reference can be the level of a biomarker detected in a healthy individual that has never had the disease. In certain embodiments, the reference can be a predetermined level of a biomarker that indicates the presence of preeclampsia in a subject. In certain embodiments, the reference can be a predetermined level of a biomarker that indicates a subject is free from preeclampsia. In certain embodiments, the reference can be an earlier sample taken from the same subject, e.g., prior to the current pregnancy or prior to having preeclampsia.

As used herein, “preeclampsia” refers to a pregnancy-associated disorder that manifests as new onset hypertension. For example, but not by way of limitation, a subject suffering from preeclampsia can have systolic blood reassure greater than 140 mmHg and a diastolic pressure greater than 90 mmHg.

As used herein, the term “biological sample” refers to a sample of biological material obtained from a subject, e.g., a human subject, including a biological fluid, e.g., blood, plasma, serum, urine, sputum, spinal fluid, pleural fluid, nipple aspirates, lymph fluid, fluid of the respiratory, intestinal, and genitourinary tracts, tear fluid, saliva, breast milk, fluid from the lymphatic system, cerebrospinal fluid, intra-organ system fluid, ascitic fluid, amniotic fluid, bronchoalveolar fluid, biliary fluid and combinations thereof. In certain non-limiting embodiments, the syncytiotrophoblast-derived microvesicles are isolated and/or purified from a blood sample obtained from a subject. In certain non-limiting embodiments, the syncytiotrophoblast-derived microvesicles are isolated and/or purified from a urine sample obtained from a subject.

The term “patient” or “subject,” as used interchangeably herein, refers to any warm-blooded animal, e.g., a human. Non-limiting examples of non-human subjects include non-human primates, dogs, cats, mice, rats, guinea pigs, rabbits, fowl, pigs, horses, cows, goats, sheep, etc. In certain embodiments, the subject is pregnant. In certain embodiments, the subject is human.

The term “microvesicle,” as used herein, refers to vesicles that are released from a cell. In certain embodiments, the microvesicle is a vesicle that is released from a cell by exocytosis of intracellular multivesicular bodies. In certain embodiments, the microvesicles can be exosomes. In certain embodiments, the microvesicles can have an average size less than about 200 nm. In certain embodiments, the microvesicles can range in size from about 30 nm to about 1000 nm.

The term “syncytiotrophoblast-derived microvesicles,” as used herein, refers to microvesicles that are derived from syncytiotrophoblasts, which are fetal-derived cells that lie at the maternal-fetal interface and tightly regulate exchange of nutrients, metabolites, and other macromolecules between the mother and the fetus.

### 2. Methods

The present disclosure provides methods for diagnosing and/or monitoring a pregnant subject with preeclampsia by analyzing microvesicles released from fetal cells, e.g., syncytiotrophoblast cells. In certain embodiments, the present disclosure provides methods for isolating, detecting, purifying, and/or analyzing microvesicles derived from syncytiotrophoblast cells (“syncytiotrophoblast-derived microvesicles”) to diagnosis a subject with preeclampsia and/or monitor a subject that has preeclampsia. In certain embodiments, the methods of the present disclosure can further include the detection and/or analysis of one or more biomarkers associated with the syncytiotrophoblast-derived microvesicles, e.g., exosomes. The biomarkers that can be used in the present disclosure are set forth below.

In certain embodiments, the information provided by the methods described herein can be used by the physician in determining the most effective course of treatment (e.g., preventative or therapeutic). A course of treatment refers to the measures taken for a patient after the diagnosis of preeclampsia is made. For example, when a subject is identified to have preeclampsia or at risk of having preeclampsia (e.g., due to having preeclampsia during an earlier pregnancy), the physician can determine whether frequent monitoring of syncytiotrophoblast-derived microvesicles and/or biomarkers associated with such microvesicles is required as a prophylactic measure.

In certain embodiments, the syncytiotrophoblast-derived microvesicles can be exosomes. In certain embodiments, the microvesicles can be in the size range from about 30 nm to 1000 nm. For example, and not by way of limitation, the microvesicles can be from about 30 nm to about 900 nm, from about 30 nm to about 800 nm, from about 30 nm to about 700 nm, from about 30 nm to about 600 nm, from about 30 nm to about 500 nm, from about 30 nm to about 400 nm, from about 30 nm to about 300 nm, from about 30 nm to about 200 nm, from about 30 nm to about 100 nm or from about 30 nm to about 50 nm in size. In certain embodiments, microvesicles can have a size range from about 30 nm to 200 nm. In certain embodiments, microvesicles can have an average size less than about 200 nm.

In certain embodiments, and as noted above, methods for assessing whether a subject suffers from preeclampsia and/or the isolation and/or purification of syncytiotrophoblast-derived microvesicles from a subject include obtaining at least one biological sample from the subject. In certain embodiments, the microvesicles can be detected in blood (including plasma or serum). Collecting a biological sample can be carried out either directly or indirectly by any suitable technique. For example, and not by way of limitation, a blood sample from a subject can be carried out by phlebotomy or any other suitable technique, with the blood sample processed further to provide a serum sample or other suitable blood fraction for analysis.

In certain embodiments, the biological sample can be obtained from the subject at any timepoint during the subject's pregnancy. For example, but not by way of limitation, the biological sample can be obtained during the first trimester, second trimester, or third trimester of the subject's pregnancy. In certain embodiments, the biological sample is obtained during the first trimester of a subject's pregnancy for early diagnosis of the subject with preeclampsia. In certain embodiments, multiple biological samples (e.g., two or more, three or more, four or more, five or more, six or more or seven or more biological samples) can be obtained during a subject's pregnancy (e.g., serially obtained samples).

### 2.1. Diagnostic Methods

In certain embodiments, methods for diagnosing a subject with preeclampsia is disclosed. In certain embodiments, the method can include (a) isolating one or more biomarkers from a biological sample of a pregnant subject; and (b) diagnosing preeclampsia in the subject, wherein the presence and/or change in the level of the one or more biomarkers indicates the presence of the pregnancy-associated disorder in the subject. In certain embodiments, the method can include (a) obtaining a biological sample from the pregnant subject; (b) isolating one or more biomarkers from the biological sample; and (c) diagnosing preeclampsia in the subject, wherein the presence and/or change in the level of the one or more biomarkers indicates the presence of the pregnancy-associated disorder in the subject. For example, but not by way of limitation, the biomarker can be syncytiotrophoblast-derived microvesicles obtained from the biological sample. In certain embodiments, a change in the size, number, and/or concentration of the isolated syncytiotrophoblast-derived microvesicles indicates the presence of preeclampsia. In certain embodiments, the biomarker can be a biomarker, e.g., protein or nucleic acid, present on the surface or within the microvesicles.

In certain embodiments, a method for diagnosing a subject with preeclampsia can include: (a) isolating one or more syncytiotrophoblast-derived microvesicles from a biological sample of a pregnant subject; (b) determining the presence and/or level of one or more biomarkers associated with the isolated syncytiotrophoblast-derived microvesicles; and (c) diagnosing preeclampsia in the subject, wherein the change in the presence and/or level of the one or more biomarkers is diagnostic of preeclampsia in the subject.

In certain embodiments, a method for diagnosing a subject with preeclampsia can include: (a) obtaining a biological sample from the subject; (b) isolating one or more syncytiotrophoblast-derived microvesicles from the biological sample; (c) determining the presence and/or level of one or more biomarkers associated with the isolated syncytiotrophoblast-derived microvesicles; and (d) diagnosing preeclampsia in the subject, wherein the change in the presence and/or level of the one or more biomarkers is diagnostic of preeclampsia in the subject.

In certain embodiments, a method for diagnosing a subject with preeclampsia can include: (a) isolating one or more syncytiotrophoblast-derived microvesicles from a biological sample of a pregnant subject; (b) determining the presence and/or level of syncytin-1 associated with the isolated syncytiotrophoblast-derived microvesicles and/or determining the number of syncytin-1-expressing syncytiotrophoblast-derived microvesicles in the biological sample; and (c) diagnosing preeclampsia in the subject, wherein a reduction in level of syncytin-1 associated with the syncytiotrophoblast-derived microvesicles and/or a reduction in the number of syncytin-1-expressing syncytiotrophoblast-derived microvesicles in the biological sample as compared to a reference control is diagnostic that the subject has preeclampsia. In certain embodiments, a method for diagnosing a subject with preeclampsia can include: (a) obtaining a biological sample from the subject; (b) isolating one or more syncytiotrophoblast-derived microvesicles from the biological sample; (c) determining the presence and/or level of syncytin-1 associated with the isolated syncytiotrophoblast-derived microvesicles and/or determining the number of syncytin-l-expressing syncytiotrophoblast-derived microvesicles in the biological sample; and (d) diagnosing preeclampsia in the subject, wherein a reduction in level of syncytin-1 associated with the syncytiotrophoblast-derived microvesicles and/or a reduction in the number of syncytin-1-expressing syncytiotrophoblast-derived microvesicles in the biological sample as compared to a reference control is diagnostic that the subject has preeclampsia. In certain embodiments, a decrease of at least about 1.5 times, at least about 2 times, at least about 2.5 times, at least about 3 times, at least about 3.5 times, at least about 4.0 times, at least about 4.5 times, or at least about 5 times in the number of syncytin-l-expressing syncytiotrophoblast-derived microvesicles as compared to a reference sample is indicative that the subject has preeclampsia. In certain embodiments, a decrease of at least about 2 times the number of syncytin-1 -expressing syncytiotrophoblast-derived microvesicles as compared to a reference sample is diagnostic that the subject has preeclampsia. In certain embodiments, a level of synctin-1 expression associated with the syncytiotrophoblast-derived microvesicles that is less than about 0.75, e.g., less than about 0.5, less than about 0.4, or less than about 0.3, as compared to a reference control is diagnostic that the subject has preeclampsia.

### 2.2. Monitoring Methods

The present disclosure further provides methods for monitoring preeclampsia in a pregnant subject. In certain embodiments, the method can include determining the level of one or more biomarkers in a biological sample obtained from the subject subsequent to a diagnosis of preeclampsia and determining the presence or level of the one or more biomarkers in a biological sample obtained from the subject at one or more later timepoints during the subject's pregnancy. In certain embodiments, a change in the level of the one or more biomarkers in the second or subsequent samples, relative to the first sample indicates that there is a change in the severity of the preeclampsia of the subject.

In certain embodiments, the present disclosure further provides methods for monitoring a subject at risk of developing preeclampsia. In certain embodiments, a subject at risk of developing preeclampsia is an individual that suffered from preeclampsia in an earlier pregnancy. For example, and not by way of limitation, the method can include determining the level of one or more biomarkers in a biological sample obtained from the pregnant subject prior to a diagnosis of preeclampsia and determining the presence or level of the one or more biomarkers in a biological sample obtained from the subject at one or more later timepoints during the subject's pregnancy. In certain embodiments, a change in the level of the one or more biomarkers in the second or subsequent samples, relative to the first sample can indicate that the subject has developed preeclampsia.

### 2.3. Methods of Treatment

The present disclosure further provides methods of treating a subject with preeclampsia. In certain embodiments, the method can include diagnosing a subject with preeclampsia as disclosed herein, followed by the treatment of the subject. For example, but not by way of limitation, a method of treatment can include: (a) obtaining a biological sample from a subject; (b) isolating one or more biomarkers from the biological sample; (c) diagnosing the subject with preeclampsia when there is a change in the presence and/or level of the one or more biomarkers as compared to a reference control level; and (d) treating the subject diagnosed with preeclampsia.

In certain embodiments, the subject can be treated by administration of medication to reduce the blood pressure of the subject, e.g., by administration of anti-hypertensive medication. In certain non-limiting embodiments, the anti-hypertensive medication is selected from the group consisting of methyldopa, labetalol, nifedipine, diazoxide, hydralazine, beta-receptor blockers, hydrochlorothiazide, and a combination thereof. In certain embodiments, the antihypertensive medication is methyldopa.

In certain embodiments, delivery can be used as a treatment for preeclampsia. Additional methods of treatment include administration of a corticosteroid and/or administration of an anti-convulsant medication. For example, but without any limitation, the anti-convulsant medication includes carbamazepine, lamotrigine, phenobarbital, valproic acid, levetiracetam, phenytoin, topiramate, valproate, benzodiazepines, polytherapy, and combination thereof. In certain embodiments, the anti-convulsant medication is carbamazepine. In certain embodiments, bed rest can be recommended for management of preeclampsia.

In certain embodiments, the methods disclosed herein can be used to monitor the response in a subject to prophylactic or therapeutic treatment (for example, treatment for preeclampsia, as disclosed above). For example, but not by way of limitation, the disclosed subject matter further provides a method of treatment including measuring the presence and/or level of one or more biomarkers of the present disclosure in a subject at a first time point, administering a therapeutic agent, re-measuring the one or more biomarkers at a second time point, comparing the results of the first and second measurements and optionally modifying the treatment regimen based on the comparison. In certain embodiments, the first time point is prior to an administration of the therapeutic agent, and the second time point is after the administration of the therapeutic agent. In certain embodiments, the first time point is prior to the administration of the therapeutic agent to the subject for the first time. In certain embodiments, the dose (defined as the quantity of therapeutic agent administered at any one administration) is increased or decreased in response to the comparison. In certain embodiments, the dosing interval (defined as the time between successive administrations) is increased or decreased in response to the comparison, including total discontinuation of treatment.

Additionally, the method of the present disclosure can be used to determine the efficacy of a disease therapy, wherein a change in the level and/or presence of a biomarker in a biological sample of a subject can indicate that the therapy regimen can be increased, maintained, reduced or stopped.

### 3. Biomarkers

In certain embodiments, the biomarker for use in the presently disclosed methods is one or more syncytiotrophoblast-derived microvesicles and/or one or more syncytiotrophoblast-derived microvesicles expressing a specific marker. For example, but not by way of limitation, the biomarker can be a pool of syncytin-l-expressing syncytiotrophoblast-derived microvesicles. In certain embodiments, a change (e.g., reduction) in a physical characteristic, e.g., number and/or concentration, or profile of the syncytiotrophoblast-derived microvesicles, e.g., syncytin-1-expressing syncytiotrophoblast-derived microvesicles, compared to a reference control is indicative of preeclampsia in a subject.

In certain embodiments, the biomarker is a protein isolated from a pool of one or more isolated syncytiotrophoblast-derived microvesicles. In certain embodiments, the disclosure provides for methods for diagnosing and/or monitoring preeclampsia in subject that include isolating syncytiotrophoblast-derived microvesicles from a biological sample of the subject, isolating one or more protein biomarkers from the syncytiotrophoblast-derived microvesicles, wherein a change in the level and/or presence of the protein biomarker compared to a reference sample is an indication that the subject has preeclampsia.

In certain embodiments, the biomarker can also be a transcribed polynucleotide or portion thereof, e.g., an mRNA or a cDNA, isolated from a pool of one or more syncytiotrophoblast-derived microvesicles. In certain embodiments, the disclosure provides for methods for diagnosing and/or monitoring preeclampsia in a subject that include isolating syncytiotrophoblast-derived microvesicles from a biological sample of the subject, isolating one or more nucleic acid biomarkers from the syncytiotrophoblast-derived microvesicles, wherein a change in the level and/or presence of the nucleic acid biomarker compared to a reference sample is an indication that the subject has preeclampsia. In certain embodiments, the nucleic acid biomarker can be mRNA. In certain embodiments, the biomarker quantitative measurement of the expression of a syncytiotrophoblast-derived microvesicle protein. Such measurements can be performed by any technique known in the art including, but not limited to, an enzyme-linked immunosorbent assay (ELISA), a nanoparticle detector-based surface marker analysis, and direct microvesicle FACS.

### 4. Microvesicle Isolation Techniques

Circulating syncytiotrophoblast-derived microvesicles can be isolated from a subject by any means known in the art and currently available. Circulating syncytiotrophoblast-derived microvesicles can be isolated from a biological sample obtained from a subject, such as a blood sample, or other biological fluid. In certain embodiments, the microvesicles can be exosomes.

There are several capture and enrichment platforms that are known in the art and currently available. For example, microvesicles can be isolated by a method of differential centrifugation as described by Raposo et al., 1996. Additional methods include anion exchange and/or gel permeation chromatography as described in U.S. Pat. Nos. 6,899,863 and 6,812,023. Methods of sucrose density gradients or Organelle electrophoresis are described in U.S. Pat. No. 7,198,923. A method of magnetic activated cell sorting (MACS) is described in Taylor and Gercel-Taylor, 2008. A method of nanomembrane ultrafiltration concentrator is described in Cheruvanky et al., 2007. Microvesicles can be identified and isolated from a biological sample of a subject by a microchip technology that uses a unique microfluidic platform to efficiently and selectively separate microvesicles (Nagrath et al., 2007). This can be adapted to identify and separate microvesicles using similar principles of capture and separation.

The microvesicles, including exosomes, can be isolated from a biological sample and analyzed using any method known in the art. In certain non-limiting embodiments, high exclusion limit agarose-based gel chromatography can be utilized to isolate microvesicles from a recipient's plasma (Taylor et al., 2005). For example, and not by way of limitation, to isolate the total vesicle fraction, the plasma sample can be fractionated using a size exclusion column, e.g., a 2.5×30cm Sepharose 2B column run isocratically with PBS, where the elution can be monitored by absorbance at 280 nm. The fractions comprising microvesicles can be concentrated using ultrafiltration with a 100K Dalton cut-off membrane. The fractions can then be ultracentrifuged, e.g., at 120,000 g for 2 hours at 4° C. to obtain a pellet that contains microvesicles.

Since microvesicles within the circulation are generated from multiple cell types, affinity-based approaches can be used to specifically purify subsets of microvesicles (Taylor et al., 2005). For immunosorbent isolation of syncytiotrophoblast-derived microvesicles, recipient plasma microvesicles can be selectively incubated with antibodies specific for syncytiotrophoblast cells, e.g., syncytin-1 or PLAP, coupled with magnetic microbeads. After incubation for 2 hours at 4° C., the magnetic bead complexes can be placed in the separator's magnetic field and the unbound microvesicles can be removed with the supernatant. The bound syncytiotrophoblast-derived microvesicles can be recovered and diluted in elution buffer (Pierce Chemical Co), centrifuged, and resuspended in PBS. Syncytiotrophoblast-derived microvesicle concentration and size distribution can be determined using the NanoSight NS300. Additional methods to isolate microvesicles include, but are not limited to, ultracentrifugation and sucrose gradient-based ultracentrifugation. In certain embodiments, the microvesicle isolation kit, EXOQUICK™, and/or the EXO-FLOW™ system from System Bioscience, Inc. can be used.

The isolation of microvesicles from syncytiotrophoblast cells can be accomplished, for example, by using antibodies, aptamers, aptamer analogs, or molecularly imprinted polymers specific for a desired surface antigen. In certain embodiments, the surface antigen is specific for syncytiotrophoblast cells. One non-limiting example of a method of microvesicle separation based on cell surface antigen is provided in U.S. Pat. No. 7,198,923. As described in, e.g., U.S. Pat. Nos. 5,840,867 and 5,582,981, WO 2003/050290 and a publication by Johnson et al., 2008, aptamers and their analogs specifically bind surface molecules and can be used as a separation tool for retrieving cell-type-specific microvesicles. Molecularly imprinted polymers also specifically recognize surface molecules as described in, e.g., U.S. Pat. Nos. 6,525,154, 7,332,553, and 7,384,589 and Bossi et al., 2007 and are a tool for retrieving and isolating cell type-specific microvesicles.

In certain embodiments, the syncytiotrophoblast-derived microvesicles can be purified, isolated, and/or identified by the detection of a cell-specific marker, e.g., specific to placental cells such as syncytiotrophoblast cells. For example, but not by way of limitation, syncytiotrophoblast-derived microvesicles can be isolated and/or identified based on the proteins residing on the surface of the microvesicles. In certain embodiments, the marker can be nucleic acids and/or proteins that reside on the surface or within the microvesicles. Non-limiting examples of such markers include syncytin-1 and placental alkaline phosphatase (PLAP). In certain embodiments, the cell-specific marker can be syncytin-1.

In certain embodiments, a method for the isolation, identification and/or purification of syncytiotrophoblast-derived microvesicles can include: (a) obtaining a biological sample from the subject; and (b) isolating, purifying and/or identifying one or more syncytiotrophoblast-derived microvesicles from the biological sample by the detection of a marker specific for syncytiotrophoblast cells, e.g., syncytin-1, placental alkaline phosphatase and/or plac-1.

In certain embodiments, a method for the isolation, identification, and/or purification of syncytiotrophoblast-derived microvesicles can include: (a) obtaining a biological sample from the subject; (b) isolating and/or purifying one or more microvesicles from the biological sample; and (c) isolating, purifying and/or identifying one or more syncytiotrophoblast-derived microvesicles from the one or more microvesicles of (b) by detecting a marker specific for syncytiotrophoblasts, e.g., syncytin-1 and placental alkaline phosphatase and/or plac-1. In certain embodiments, isolating and/or purifying one or more microvesicles from the biological sample can include the detection of one or more canonical markers specific for microvesicles, which can include, but are not limited to, flotillin-1, CD63, CD9, CD81, and/or TSG101.

In certain embodiments, the present disclosure provides a method for preparing a syncytiotrophoblast-derived microvesicles fraction from a pregnant subject useful for detection of preeclampsia. In certain embodiments, the method can include: (a) extracting one or more microvesicles from a biological sample of a pregnant subject; (b) producing a fraction of the microvesicles extracted in (a) by selectively enriching syncytiotrophoblast-derived microvesicles; and (c) measuring one or more biomarkers. For example, but not by way of limitation, the fraction of syncytiotrophoblast-derived microvesicles can be produced by contacting the sample with an agent binding a cell-specific marker, e.g., specific to placental cells such as syncytiotrophoblast cells, and/or a protein residing on the surface of the microvesicles. Additional non-limiting examples of cell-specific markers include syncytin-1, placental alkaline phosphatase (PLAP), plac-1, flotillin-1, CD63, CD9, CD81 and/or TSG101. In certain embodiments, the cell-specific marker can be syncytin-1.

In certain embodiments, the method for preparing the syncytiotrophoblast-derived microvesicles fraction can include size discrimination of microvesicles. In certain non-limiting embodiments, the method can discriminate microvesicles in the size range from about 30 nm to about 1000 nm. For example, and not by way of limitation, the microvesicles can be from about 30 nm to about 900 nm, from about 30 nm to about 800 nm, from about 30 nm to about 700 nm, from about 30 nm to about 600 nm, from about 30 nm to about 500 nm, from about 30 nm to about 400 nm, from about 30 nm to about 300 nm, from about 30 nm to about 200 nm, from about 30 nm to about 100 nm or from about 30 nm to about 50 nm in size. In certain embodiments, microvesicles can have a size range from about 30 nm to about 200 nm. In certain embodiments, microvesicles can have an average size less than about 200 nm.

### 5. Biomarker Detection

Biomarkers used in the methods of the present disclosure can be identified in a biological sample using any method known in the art. Biomarkers can be syncytiotrophoblast-derived microvesicles and nucleic acids and/or proteins that reside on the surface or within the syncytiotrophoblast-derived. The syncytiotrophoblast-derived, e.g., exosomes, can be isolated from a biological sample and analyzed using any method known in the art. The nucleic acid sequences, fragments thereof, and proteins, and fragments thereof, can be isolated and/or identified in a biological sample using suitable methods known in the art.

### 5.1. Protein Detection Techniques

In certain embodiments, the methods of the present disclosure can include the detection of one or more markers specific to syncytiotrophoblast cells to confirm the proper isolation and/or purification of syncytiotrophoblast-derived microvesicles. In certain embodiments, the marker for syncytiotrophoblast-derived microvesicles can be a protein present on the surface and/or within the syncytiotrophoblast-derived microvesicles, e.g., exosomes. In certain embodiments, such a marker can be used a biomarker to diagnose a subject with a disease and/or disorder or monitor a subject with a disease and/or disorder, e.g., preeclampsia.

Proteins can be isolated from a microvesicle using any number of methods, which are well-known in the art, the particular isolation procedure chosen being appropriate for the particular biological sample. In certain embodiments, the protein can be detected on the surface of the microvesicle.

Methods for the detection of proteins are well known to those skilled in the art, and include but are not limited to mass spectrometry techniques, 1-D or 2-D gel-based analysis systems, chromatography, enzyme linked immunosorbent assays (ELISAs), radioimmunoassays (RIA), enzyme immunoassays (EIA), Western Blotting, immunoprecipitation and immunohistochemistry. These methods use antibodies, or antibody equivalents, to detect protein. Antibody arrays or protein chips can also be employed, see for example U.S. Patent Application Nos: 2003/0013208A1; 2002/0155493A1, 2003/0017515 and U.S. Pat. Nos. 6,329,209 and 6,365,418, herein incorporated by reference in their entirety.

ELISA and RIA procedures can be conducted such that a protein standard is labeled (with a radioisotope such as ¹²⁵I or ³⁵S, or an assayable enzyme, such as horseradish peroxidase or alkaline phosphatase), and, together with the unlabeled sample of microvesicles, brought into contact with the corresponding antibody, whereon a second antibody is used to bind the first, and radioactivity or the immobilized enzyme assayed (competitive assay). Alternatively, the protein present on and/or within the microvesicles can react with the corresponding immobilized antibody, radioisotope, or enzyme-labeled anti-marker antibody is allowed to react with the system, and radioactivity or the enzyme assayed (ELISA-sandwich assay). Other conventional methods can also be employed as suitable.

The above techniques can be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods can also be employed as suitable.

In certain embodiments, the detection of a protein marker from a syncytiotrophoblast-derived microvesicle sample includes contacting the microvesicle sample with an antibody or variant (e.g., fragment) thereof which selectively binds the protein marker, and detecting whether the antibody or variant thereof is bound to the sample. The method can further include contacting the sample with a second antibody, e.g., a labeled antibody. The method can further include one or more washing, e.g., to remove one or more reagents.

It can be desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene can provide a suitable support.

Enzymes employable for labeling are not particularly limited but can be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase can be assayed by measuring the concentration of hydrogen peroxide formed after reaction of the enzyme-labeled antibody with the substrate under controlled conditions well-known in the art.

Other techniques can be used to detect a protein marker according to a practitioner's preference based upon the present disclosure. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Antibodies (unlabeled) are then brought into contact with the support and assayed by a secondary immunological reagent, such as labeled protein A or anti-immunoglobulin (suitable labels including ¹²⁵I, horseradish peroxidase, and alkaline phosphatase). Chromatographic detection can also be used.

Other machine or autoimaging systems can also be used to measure immunostaining results for the marker. As used herein, “quantitative” immunohistochemistry refers to an automated method of scanning and scoring samples that have undergone immunohistochemistry, to identify and quantitate the presence of a specified marker, such as an antigen or other protein. The score given to the sample is a numerical representation of the intensity of the immunohistochemical staining of the sample and represents the amount of target marker present in the sample. As used herein, Optical Density (OD) is a numerical score that represents intensity of staining. As used herein, semi-quantitative immunohistochemistry refers to scoring of immunohistochemical results by human eye, where a trained operator ranks results numerically (e.g., as 1, 2 or 3).

Various automated sample processing, scanning, and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Another method that can be used for detecting protein markers is Western blotting. Immunodetection can be performed with antibody to a protein marker using the enhanced chemiluminescence system (e.g., from PerkinElmer Life Sciences, Boston, Mass.). The membrane can then be stripped and re-blotted with a control antibody, e.g., anti-actin.

Antibodies against protein markers can also be used for imaging purposes, for example, to detect the presence of syncytiotrophoblast-derived microvesicles in a sample of microvesicles obtained from a recipient's blood. Suitable labels include radioisotopes, iodine (¹²⁵¹ 1) carbon (¹⁴C) sulfur (³⁵S), tritium (³H), indium (¹¹²In), and technetium (^{99m}Tc), fluorescent labels, such as fluorescein, rhodamine, and biotin. Immunoenzymatic interactions can be visualized using different enzymes such as peroxidase, alkaline phosphatase, or different chromogens such as DAB, AEC or Fast Red.

For in vivo imaging purposes, antibodies are not detectable, as such, from outside the body, and so must be labeled, or otherwise modified, to permit detection. Labels for this purpose can be any that do not substantially interfere with the antibody binding, but which allow external detection. Suitable labels can include those that can be detected by X-radiography, NMR or MRI. For X-radiographic techniques, suitable labels include any radioisotope that emits detectable radiation but that is not overtly harmful to the subject, such as barium or cesium, for example. Suitable labels for NMR and MRI generally include those with a detectable characteristic spin, such as deuterium, which can be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99 m.

The labeled antibody or antibody fragment will then preferentially accumulate at the location of the sample which contains a protein marker. The labeled antibody or variant thereof, e.g., antibody fragment, can then be detected using known techniques. Antibodies for use in the present disclosure include any antibody, whether natural or synthetic, full length or a fragment thereof, monoclonal or polyclonal, that binds sufficiently strongly and specifically to the marker to be detected. An antibody can have a K_{d} of at most about 10⁻⁶M, 10⁻⁷M, 10⁻⁸M, 10⁻⁹M, 10⁻¹⁰M, 10⁻¹¹, 10⁻¹²M. The phrase “specifically binds” refers to binding of, for example, an antibody to an epitope or antigen or antigenic determinant in such a manner that binding can be displaced or competed with a second preparation of identical or similar epitope, antigen, or antigenic determinant.

Antibodies and derivatives thereof that can be used encompasses polyclonal or monoclonal antibodies, chimeric, human, humanized, primatized (CDR-grafted), veneered or single-chain antibodies, phase produced antibodies (e.g., from phage display libraries), as well as functional binding fragments, of antibodies. For example, antibody fragments capable of binding to a marker, or portions thereof, including, but not limited to Fv, Fab, Fab′ and F(ab′)₂ fragments can be used. Such fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH, domain and hinge region of the heavy chain. In certain embodiments, the antibodies can be conjugated to quantum dots.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies.

In certain embodiments, agents that specifically bind to a polypeptide other than antibodies are used, such as peptides. Peptides that specifically bind can be identified by any means known in the art, e.g., peptide phage display libraries. Generally, an agent that is capable of detecting a marker polypeptide, such that the presence of a marker is detected and/or quantitated, can be used. As defined herein, an “agent” refers to a substance that is capable of identifying or detecting a protein marker in a sample (e.g., identifies or detects the mRNA of a marker, the DNA of a marker, the protein of a marker). In certain embodiments, the agent is a labeled or labelable antibody which specifically binds to a marker polypeptide.

In addition, a protein marker can be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See, for example, U.S. Patent Application Nos: 2003/0199001, 2003/0134304, 2003/0077616, which are herein incorporated by reference.

Mass spectrometry methods are well known in the art and have been used to detect biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer can be used. In other embodiments, laser-desorption/ionization mass spectrometry is used to analyze the sample. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

For additional information regarding mass spectrometers, see, e.g., Principles of Instrumental Analysis, 3rd edition. Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

Detection of the presence of a marker or other substances can involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of a particular marker. Software programs such as the Biomarker Wizard program (Ciphergen Biosystems, Inc., Fremont, Calif) can be used to aid in analyzing mass spectra. The mass spectrometers and their techniques are well known to those of skill in the art.

Any person skilled in the art understands, the components of a mass spectrometer (e.g., desorption source, mass analyzer, detect, etc.) and varied sample preparations can be combined with other suitable components or preparations described herein, or to those known in the art. For example, in certain embodiments, a control sample can contain heavy atoms (e.g., 13C) thereby permitting the test sample to be mixed with the known control sample in the same mass spectrometry run.

In certain embodiments, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

5.2. RNA Detection Techniques

In certain embodiments, the methods of the present disclosure can include the detection of one or more markers specific to syncytiotrophoblast cells to confirm the proper isolation and/or purification of syncytiotrophoblast-derived microvesicles. In certain embodiments, such a marker can be used as a biomarker to diagnose a subject with a disease and/or disorder or monitor a subject with a disease and/or disorder, e.g., preeclampsia.

In certain embodiments, the marker is a nucleic acid, including DNA and/or RNA, contained within the syncytiotrophoblast-derived microvesicles, e.g., exosomes. Nucleic acid molecules can be isolated from a microvesicle using any number of methods, which are well-known in the art, the particular isolation procedure chosen being appropriate for the particular biological sample. In certain instances, with some techniques, it can also be possible to analyze the nucleic acid without extraction from the microvesicle.

In certain embodiments, the detection of nucleic acids present in the microvesicles can be quantitative and/or qualitative. Any method for qualitatively or quantitatively detecting a nucleic acid marker can be used. Detection of RNA transcripts can be achieved, for example, by Northern blotting, wherein a preparation of RNA is run on a denaturing agarose gel, and transferred to a suitable support, such as activated cellulose, nitrocellulose, or glass or nylon membranes. Radiolabeled cDNA or RNA is then hybridized to the preparation, washed, and analyzed by autoradiography.

Detection of RNA transcripts can further be accomplished using amplification methods. For example, it is within the scope of the present disclosure to reverse transcribe mRNA into cDNA followed by polymerase chain reaction (RT-PCR); or, to use a single enzyme for both as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). In certain embodiments, quantitative real-time polymerase chain reaction (qRT-PCR) can be used to detect RNA.

Other known amplification methods which can be utilized herein include, but are not limited to, the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO9322461.

In situ hybridization visualization can also be employed. Another method for detecting mRNAs in a microvesicle sample is to detect mRNA levels of a marker by fluorescent in situ hybridization (FISH). FISH is a technique that can directly identify a specific sequence of DNA or RNA in a cell, microvesicle sample, or biological sample and therefore enables to visual determination of the marker presence and/or expression in tissue samples. Fluorescence in situ hybridization is a direct in situ technique that is relatively rapid and sensitive. FISH test also can be automated. Immunohistochemistry can be combined with a FISH method when the expression level of the marker is difficult to determine by immunohistochemistry alone.

Alternatively, RNA can be detected on a DNA array, chip, or a microarray. Oligonucleotides corresponding to the marker(s) are immobilized on a chip which is then hybridized with labeled nucleic acids of a test sample obtained from a subject. Positive hybridization signal is obtained with the sample containing marker transcripts. Methods of preparing DNA arrays and their use are well known in the art. (See, for example, U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

To detect RNA molecules, for example, mRNA can be extracted from the microvesicle sample to be tested, reverse transcribed and fluorescent-labeled cDNA probes are generated. Using microarrays capable of hybridizing to a marker, cDNA can be probed with the labeled cDNA probes, and they can be slides scanned and the fluorescence intensity measured. This intensity correlates with the hybridization intensity and expression levels.

Types of probes for detection of RNA include cDNA, riboprobes, synthetic oligonucleotides and genomic probes. The type of probe used will generally be dictated by the particular situation, such as riboprobes for in situ hybridization, and cDNA for Northern blotting, for example. In certain embodiments, the probe is directed to nucleotide regions unique to the particular marker RNA. The probes can be as short as is required to differentially recognize the particular marker RNA transcripts, and can be as short as, for example, 15 bases; however, probes of at least 17 bases, e.g., 18 bases or 20 bases can be used. In certain embodiments, the primers and probes hybridize specifically under stringent conditions to a nucleic acid fragment having the nucleotide sequence corresponding to the target gene. As herein used, the term “stringent conditions” means hybridization will occur only if there is at least 95% and at least 97% identity between the sequences.

The form of labeling of the probes can be any that is appropriate, such as the use of radioisotopes, for example, ³²P and ³⁵S. Labeling with radioisotopes can be achieved, whether the probe is synthesized chemically or biologically, by the use of suitably labeled bases.

### 6. Kits

The present disclosure further provides kits for diagnosing and/or monitoring a subject with preeclampsia that provides for isolating, purifying, and/or detecting one or more syncytiotrophoblast-derived microvesicles. In certain embodiments, the kit can include one or more provisions for detecting one or more markers, e.g., biomarkers, present on the surface of the syncytiotrophoblast-derived microvesicles or present within the syncytiotrophoblast-derived microvesicles. In certain embodiments, a kit of the present disclosure can further include one or more markers for isolating microvesicles from a biological sample.

Types of kits include, but are not limited to, packaged probe and primer sets (e.g., TaqMan probe/primer sets), arrays/microarrays, syncytiotrophoblast marker-specific antibodies, and antibody-conjugated beads or quantum dots, which further contain one or more probes, primers, or other detection reagents for isolating and/or detecting one or more microvesicles and/or one or more syncytiotrophoblast-derived microvesicles, disclosed herein. For example, but not by way of limitation, the syncytiotrophoblast marker can include syncytin-1, plac-1, and/or PLAP. In certain embodiments, the marker specific for microvesicles can include flotillin-1 and/or CD63.

In certain non-limiting embodiments, a kit can include a pair of oligonucleotide primers suitable for polymerase chain reaction (PCR) or nucleic acid sequencing, for detecting one or markers of the syncytiotrophoblast-derived microvesicles. A pair of primers can include nucleotide sequences complementary to a marker and be of sufficient length to selectively hybridize with said marker. Alternatively, the complementary nucleotides can selectively hybridize to a specific region in close enough proximity 5′ and/or 3′ to the marker position to perform PCR and/or sequencing. Multiple marker-specific primers can be included in the kit to simultaneously assay large number of markers. The kit can also comprise one or more polymerases, reverse transcriptase, and nucleotide bases, wherein the nucleotide bases can be further detectably labeled.

In certain non-limiting embodiments, a primer can be at least about 10 nucleotides or at least about 15 nucleotides or at least about 20 nucleotides in length and/or up to about 200 nucleotides or up to about 150 nucleotides or up to about 100 nucleotides or up to about 75 nucleotides or up to about 50 nucleotides in length.

In certain non-limiting embodiments, the oligonucleotide primers can be immobilized on a solid surface or support, for example, on a nucleic acid microarray, wherein the position of each oligonucleotide primer bound to the solid surface or support is known and identifiable.

In certain non-limiting embodiments, a kit can comprise at least one nucleic acid probe, suitable for in situ hybridization or fluorescent in situ hybridization, for detecting the marker and/or the syncytiotrophoblast-derived microvesicles. Such kits will generally comprise one or more oligonucleotide probes that have specificity for various markers.

In certain non-limiting embodiments, a kit can comprise at least one antibody for immunodetection of the marker and/or the syncytiotrophoblast-derived microvesicles and/or for the isolation of the syncytiotrophoblast-derived microvesicles. Antibodies, both polyclonal and monoclonal, specific for a marker, can be prepared using conventional immunization techniques, as will be generally known to those of skill in the art. The immunodetection reagents of the kit can include detectable labels that are associated with, or linked to, the given antibody or antigen itself Such detectable labels include, for example, chemiluminescent or fluorescent molecules (rhodamine, fluorescein, green fluorescent protein, luciferase, Cy3, Cy5, or ROX), radiolabels (3H, 35S, 32P, 14C, 1311), quantum dots or enzymes (alkaline phosphatase, horseradish peroxidase).

In certain non-limiting embodiments, the antibody can be provided bound to a solid support, such as a column matrix, an array, or well of a microtiter plate. Alternatively, the support can be provided as a separate element of the kit.

In certain non-limiting embodiments, a kit can comprise one or more primers, probes, microarrays, or antibodies suitable for detecting one or more markers.

In certain non-limiting embodiments, where the measurement means in the kit employs an array, the set of markers set forth above can constitute at least 10 percent or at least 20 percent or at least 30 percent or at least 40 percent or at least 50 percent or at least 60 percent or at least 70 percent or at least 80 percent of the species of markers represented on the microarray.

In certain non-limiting embodiments, a marker detection kit can comprise one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction to detect a marker. A kit can also include additional components or reagents necessary for the detection of a marker, such as secondary antibodies for use in immunohistochemistry. A kit can further include one or more other markers or reagents for evaluating other prognostic factors, e.g., stage of rejection.

In certain non-limiting embodiments, a marker detection kit can comprise one or more reagents and/or tools for isolating syncytiotrophoblast-specific microvesicles from a biological sample. A kit can also include reagents necessary for isolating the protein and/or nucleic acids from the isolated microvesicles.

### 7. Reports, Programmed Computers, and Systems

In certain embodiments, the diagnosis and/or monitoring of preeclampsia in a subject based on the isolation, purification, and/or detection of syncytiotrophoblast-derived microvesicles, can be referred to herein as a “report.” A tangible report can optionally be generated as part of a testing process (which can be interchangeably referred to herein as “reporting,” or as “providing” a report, “producing” a report, or “generating” a report).

Examples of tangible reports can include, but are not limited to, reports in paper (such as computer-generated printouts of test results) or equivalent formats and reports stored on computer readable medium (such as a CD, USB flash drive or other removable storage device, computer hard drive, or computer network server, etc.). Reports, particularly those stored on computer readable medium, can be part of a database, which can optionally be accessible via the internet (such as a database of patient records or genetic information stored on a computer network server, which can be a “secure database” that has security features that limit access to the report, such as to allow only the patient and the patient's medical practitioners to view the report while preventing other unauthorized individuals from viewing the report, for example). In addition to, or as an alternative to, generating a tangible report, reports can also be displayed on a computer screen (or the display of another electronic device or instrument).

A report can include, for example, an individual's medical history, or can just include size, presence, absence or levels of one or more markers (for example, a report on computer readable medium such as a network server can include hyperlink(s) to one or more journal publications or websites that describe the medical/biological implications). Thus, for example, the report can include information of medical/biological significance as well as optionally also including information regarding the detection of syncytiotrophoblast-derived microvesicles, or the report can just include information regarding the detection of syncytiotrophoblast-derived microvesicles without other medical/biological significance.

A report can further be “transmitted” or “communicated” (these terms can be used herein interchangeably), such as to the individual who was tested, a medical practitioner (e.g., a doctor, nurse, clinical laboratory practitioner, genetic counselor, etc.), a healthcare organization, a clinical laboratory, and/or any other party or requester intended to view or possess the report. The act of “transmitting” or “communicating” a report can be by any means known in the art, based on the format of the report. Furthermore, “transmitting” or “communicating” a report can include delivering a report (“pushing”) and/or retrieving (“pulling”) a report. For example, reports can be transmitted/communicated by various means, including being physically transferred between parties (such as for reports in paper format) such as by being physically delivered from one party to another, or by being transmitted electronically or in signal form (e.g., via e-mail or over the internet, by facsimile, and/or by any wired or wireless communication methods known in the art) such as by being retrieved from a database stored on a computer network server, etc.

In certain embodiments, the disclosed subject matter provides computers (or other apparatus/devices such as biomedical devices or laboratory instrumentation) programmed to carry out the methods described herein. In certain embodiments, the system can be controlled by the individual and/or their medical practitioner in that the individual and/or their medical practitioner requests the test, receives the test results back, and (optionally) acts on the test results to reduce the individual's disease risk, such as by implementing a disease management system.

### EXAMPLES

The following example is offered to more fully illustrate the present disclosure but is not to be construed as limiting the scope thereof.

### Example 1: Syncytiotrophoblast Extracellular Microvesicle Profiles in Maternal Circulation for Noninvasive Diagnosis of Preeclampsia

Placental trophoblasts, especially syncytiotrophoblasts, are fetal-derived cells that lie at the maternal-fetal interface and tightly regulate exchange of nutrients, metabolites, and other macromolecules between these two entities. Several studies have shown that syncytiotrophoblasts are highly active in fetal physiology, and their dysfunction has been implicated in placental pathophysiology (Mitchell et al., 2015). In addition, syncytiotrophoblasts also release a wide array of extracellular vesicles into the maternal circulation throughout gestation (Germain et al., 2007, Mitchell et al., 2015, Sarker et al., 2014, Tannetta et al., 2017a, Tannetta et al., 2017b), with increasing amounts seen during the latter stages of pregnancy. These include apoptotic bodies, microvesicles such as ectosomes and microparticles, and extracellular microvesicles (EVs) including exosomes, which are released via different mechanistic processes and are detectable in peripheral blood as microvesicles of different sizes. Although the exact definition of these microvesicles by size remains somewhat nebulous, there is excellent consensus that exosomes are nanoparticles in the range of 30 to 200 nm that are specifically released through endosomal pathway utilizing endocytic machinery via subcellular structures called multivesicular bodies (Lotvall et al., 2014).

Exosomes express canonical and tissue specific proteins on their surface, and their intraexosomal compartments are enriched in functional macromolecules implicated to have paracrine and systemic effects on target tissues (Lotvall et al., 2014, Valadi et al., 2007). Recently, several groups have investigated the diagnostic and physiologic implications of maternal plasma microvesicles, including EVs, in normal pregnancy and in pregnancy-associated disorders. These studies suggest that in addition to changes in total maternal plasma microvesicle pools, selective expression levels of circulating placental proteins may also be altered in conditions of normal pregnancy versus pregnancy-associated disorders such as preeclampsia. In this context, three placental proteins, placental alkaline phosphatase (PLAP), syncytin-1 and syncytin-2 have been studied (Cuffe et al., 2017, Mitchell et al., 2015, Pillay et al., 2016, Salomon et al., 2017, Salomon and Rice, 2017, Tan et al., 2014, Vargas et al., 2009, Vargas et al., 2011, Vargas et al., 2014). These studies showed alterations in placental protein levels in EVs isolated from maternal plasma samples from subjects with preeclampsia versus normal pregnancy, although with some mixed results. But overall, the studies validate that placental dysfunction is associated with altered circulating placental microvesicles/placental proteins in maternal plasma, and therefore, characterization of their profiles has diagnostic potential for noninvasive detection and monitoring of pregnancy-associated disorders.

### Materials and Methods:

Design and subject samples. The present disclosure utilizes blood samples that were collected from women enrolled in a prospective longitudinal evaluation evaluating biomarker differences in women with and without severe Preeclampsia. Women were enrolled from April 2015 to May 2017 at the Hospital of the University of Pennsylvania after obtaining written informed consent. Institutional Review Board approval was obtained prior to initiation. Cases were women diagnosed with preterm (23-36 6/7 weeks) preeclampsia with severe features who were admitted to the Obstetrical unit at the hospital. Preeclampsia with severe features was defined by current guidelines from the Hypertension Task Force of the American College of Obstetricians and Gynecologists (American College of et al., 2013). Normotensive controls were recruited in the outpatient setting and matched by gestational age of Preeclampsia diagnosis (±3 weeks), race, maternal age (±8 years) and body mass index (±5 kg/m²). Blood was drawn at the time of enrollment for both cases and controls. Women were followed prospectively into the postpartum period. Controls who developed any form of pregnancy related hypertension were subsequently excluded post enrollment. Women with preexisting cardiovascular disease were excluded. Baseline patient demographics and characteristics are summarized in Table 1.

TABLE 1
Demographics and patient characteristics.

| | Case (n = 21) | Control (n = 23) | P value |
|---|---|---|---|
| Maternal age- | 29.7 ± 8.2 | 26.4 ± 5.1 | 0.12 |
| mean ± SD (years) | | | |
| Race-n (%) | | | |
| African American | 19 (90.5) | 20 (87.0) | 0.71 |
| White | 2 (9.5) | 3 (13.0) | |
| Body Mass Index- | 31.2 ± 7.5 | 25.6 ± 6.2 | 0.01 |
| mean ± SD (kg/m2) | | | |
| Gestational age at | 31.3 ± 4.4 | 33.2 ± 4.0 | 0.63 |
| blood draw- | | | |
| mean ± SD (weeks) | | | |
| Parity-median [IQR] | 0 [0-1] | 1 [0-1] | 0.46 |
| Tobacco use-n (%) | 0 (0) | 2 (8.7) | 0.43 |
| Chronic HTN-n (%) | 12 (57.1) | 1 (4.3) | 0.02 |

BeWo cell line and EV isolation. To validate the presence of syncytiotrophoblast specific markers on EV surfaces, these markers were first analyzed for in an in vitro system. Human choriocarcinoma-derived cell line (BeWo) was grown in Dulbecco's modified Eagle's medium (DMEM) (with L-glutamine and 4500 mg glucose/L, without sodium bicarbonate; Sigma Chemical Co., Missouri, cat. no. D-5648), heat-inactivated fetal bovine serum (FBS) (Atlanta Biologicals, Georgia) and penicillin/streptomycin (10,000 U/mL; Invitrogen, California) until 90% confluent.

Culture medium was replaced with exosome free FBS and cells were grown for another 48 hours. Culture medium was collected and EVs were isolated using methodology of ultracentrifugation. First, culture medium was spun at 800 g for 5 minutes followed by 2000 g for 10 minutes to remove cellular debris. Supernatant was collected and ultrafiltered using a 100 kDa cut-off membrane, followed by ultracentrifugation at 120,000 g for 2 hours at 4° C. Pellets containing EVs were resuspended in 1× phosphate buffered saline (PBS).

Plasma EMV isolation. Blood was collected from all patients in EDTA tubes and was immediately spun down at 2,000 g for 15 minutes at 4° C. Plasma was removed and aliquoted into 250 μL portions. The plasma was frozen at −80° C. until all samples were collected in order to isolate all EVs at the same time. 250 μL of human plasma was passed through size exclusion chromatography column to obtain eluent fractions containing EVs (Vallabhajosyula et al., 2017). The pooled fractions were filtered through a 100 kDa cut-off membrane (Thermo Fisher Scientific, Waltham, Mass.) and ultracentrifuged at 120,000 g for 2 hours at 4° C. The spun-down pellet containing EVs was resuspended in 400 μL of phosphate buffered saline (1× PBS) for downstream analysis.

Nanoparticle detector analysis. Isolated EVs from maternal plasma and BeWo cell line EVs were analyzed on the NanoSight NS300 nanoparticle detector light scatter mode (Malvern Instruments Inc., Massachusetts) for quantitation and size distribution of EVs. All captures were taken at a camera level of 16 with a detection threshold of 10. For STEV subpopulation analysis, EVs were studied for surface expression of synctin-1, PLAP, and PLAC-1 using anti-human syncytin-1 (Santa Cruz Biotechnology, California), anti-PLAP (Santa Cruz Biotechnology, California) and anti-PLAC-1 (Santa Cruz Biotechnology, California) conjugated quantum dots (Thermo Fisher Scientific, Massachusetts) on the nanoparticle detector fluorescence mode. Rabbit IgG, mouse IgG and goat IgG antibody quantum dot (Santa Cruz, California) were used as isotype controls. Each sample was run in duplicates and each experimental run was duplicated independently; the mean value of the two independent runs is represented. In each displayed panel, the nanoparticle size distribution curve is represented by particle size (nanometers) on the x-axis and nanoparticle concentration (x10⁶/ml) on the y-axis. The full curve represents total plasma EV pool distribution, and empty curve represents the respective subpopulation.

Western blot analysis. BeWo cells were lysed using radioimmunoprecipitation assay (RIPA) buffer (Thermo Fisher Scientific, Massachusetts). Cell lysates were used as a positive control and as tissue confirmation of the protein markers being assessed. Equal quantities of EV protein and cell lysate (10 μg) were run on polyacrylamide gels and then transferred onto nitrocellulose membranes (Life Technologies, New York). After blocking with 5% milk, membranes were probed with primary antibodies specific to the following proteins: syncytin-1 (1:500, Santa Cruz Biotechnology, D, PLAP (1:200, Santa Cruz Biotechnology, California), Plac-1 (1:200, Santa Cruz Biotechnology, California), CD63 (1:1000, Santa Cruz Biotechnology, California), Cytochrome C (1:500, Santa Cruz Biotechnology, California), β-actin (1:200, Proteintech, Illinois), and flotillin-1 (1:1000, Proteintech, Illinois). Horseradish peroxidase coupled secondary antibodies were added and detected through chemiluminescence using ImageQuant LAS 400 phosphoimager.

Statistical analysis. First, data was checked for distribution. Comparative analysis for continuous variables (total EV concentration and STEV signal for Preeclampsia versus healthy pregnant control) were performed using independent sample t-test (2-tailed).

For receiver operating characteristic (ROC) curve, the true-positive rate (sensitivity) was plotted against the false-positive rate (specificity) to illustrate performance of a binary classifying system (Control versus Preeclampsia groups). A threshold was determined using the Youden index, and likelihood ratio, sensitivity and specificity were calculated. ROC curves were compared using the method of Delong et al (DeLong et al., 1988). General statistics were assessed using StataMP version 14.2 (StataCorp LP, Texas), and scatter plots and NanoSight panels were constructed using Prism version 7.0 (GraphPad, California). All reported tests were 2-tailed and alpha level was set to 0.05.

### Results:

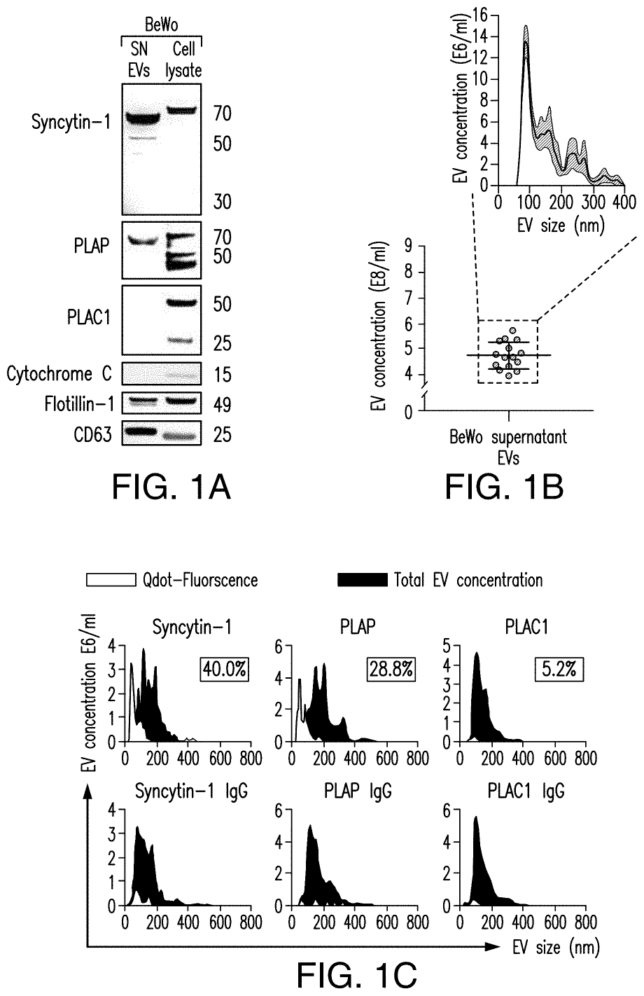

EVs released by BeWo cells express placenta specific proteins. EV fraction obtained from BeWo culture supernatant was confirmed by Western blot for expression of canonical exosome markers such as flotillin-1, CD63, and for minimal contamination with other microvesicles such as apoptotic bodies (cytochrome C) (FIG. 1A). A shown in FIG. 1A, supernatant EVs were positive for canonical exosomal markers CD63 and flotillin-1, suggesting that EVs isolated utilizing methodology detailed yielded nanoparticles enriched in exosomes. There was no contamination from cellular debris and apoptotic bodies in EV fractions, as evidenced by the absence of cytochrome C. BeWo EVs showed high expression of placental proteins syncytin-1 and PLAP, but not of PLAC-1. Expression of 3 placenta-specific proteins, syncytin-1, PLAP and plac-1 were then analyzed. BeWo EVs showed high expression of syncytin-1 and PLAP, but no expression of plac-1 (FIG. 1A). Nanoparticle analysis of EV preparations of supernatant samples from different BeWo cell cultures showed similar size distribution, with majority of the EVs in the size range consistent with exosomes (<200 nm) (FIG. 1B). EVs were next studied on the nanoparticle detector for surface expression of placental proteins (FIG. 1C). A higher abundance of placenta-specific markers syncytin-1 and PLAP was noted compared to PLAC-1, which was not elevated compared to IgG isotype background fluorescence. As shown in FIG. 1C, EV subpopulations positive for syncytin-1, PLAP and PLAC-1 (empty) are shown in relation to the total EV pool (full). Appropriate IgG isotypes (mouse IgG, rabbit IgG) were used as negative controls. Syncytin-1 and PLAP expression was noted at high levels, compared to plac-1. Taken together, these studies suggested that trophoblast cells release EVs that express placental proteins on their surface. Given the literature supporting expression of PLAP and syncytin-1 in maternal plasma during pregnancy, the utility of syncytin-1 as a diagnostic marker for syncytiotrophoblast EVs in maternal plasma was further studied.

Patient clinical data. Demographics, clinical parameters, and neonatal data are shown in Table 1 above. Subjects with preeclampsia were older (29.7±8.2 years versus 26.4±5.1 years; p=0.12) and had higher body mass index (31.2±7.5 kg/m² versus 25.6±6.2 kg/m²; p=0.01).

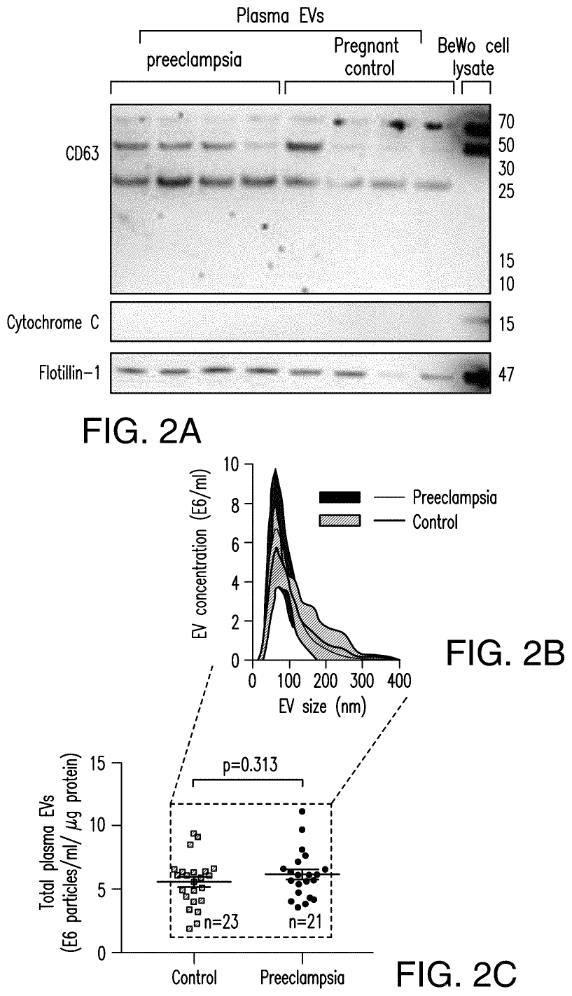

Maternal plasma EV analysis. First, the EVs isolated using the methodology detailed yielded nanoparticles enriched in exosome markers (FIG. 2A), with minimal contamination from cellular debris and apoptotic bodies. Further, nanoparticle analysis showed that the majority of isolated EVs had size distribution consistent with exosomes, with peak distribution at <100 nm (FIG. 2B). Next, whether total EV quantities were altered in normal pregnancy versus preeclampsia was analyzed. There was no statistical difference in total EV quantities in normal pregnancy controls versus preeclampsia subjects (p=0.313, FIG. 2B).

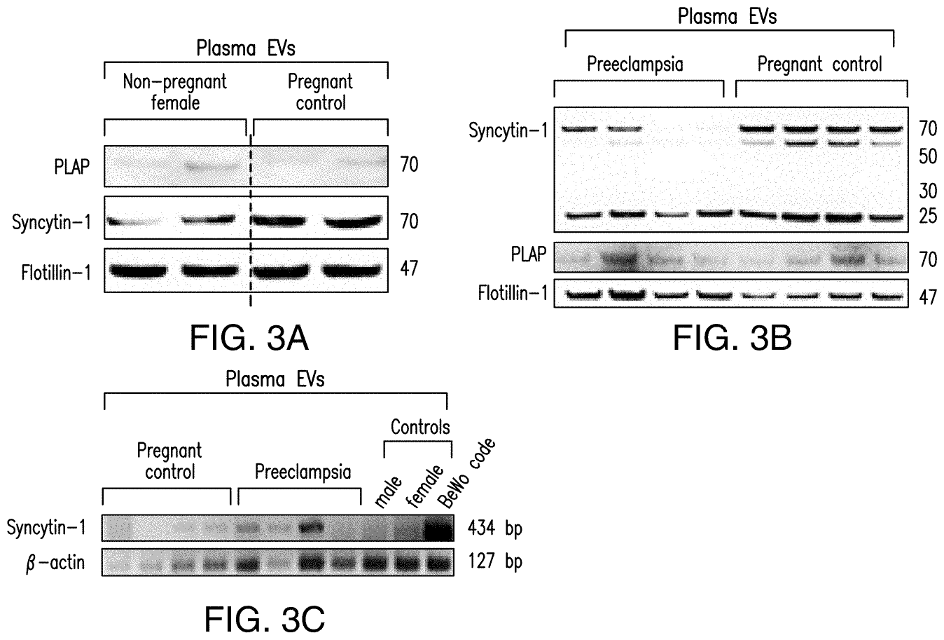

Given these findings, whether plasma EVs express syncytiotrophoblast protein markers was assessed next. On Western blot, PLAP detection was noted in maternal plasma EV samples from control and preeclampsia groups. Furthermore, there was also detection of PLAP in non-pregnant females, suggesting that antibody was possibly cross reacting to alkaline phosphatase isoforms released by other tissue types (FIG. 3A). As shown in FIG. 3A, similar levels of PLAP were seen in non-pregnant female controls compared to pregnant women. Presence of flotillin-1 suggested that EVs isolated utilizing methodology detailed yielded nanoparticles enriched in exosomes. Next, syncytin-1 expression was tested. Compared to male and non-pregnant female controls, which showed very low expression of syncytin-1, maternal plasma EVs from control subjects showed high syncytin-1 expression by Western blot (FIG. 3B). This difference was more pronounced with syncytin-1 than PLAP. In addition, subjects from the preeclampsia group showed decreased syncytin-1 expression (FIG. 3B). As shown in FIG. 3B, similar levels of PLAP were seen in preeclampsia versus control samples. Lower levels of syncytin-1 were seen in preeclampsia subjects compared to healthy pregnant controls. Canonical exosome marker, flotillin-1, is shown as positive control. Therefore, syncytin-1 levels in the plasma EV pools suggested decreased expression in preeclampsia subjects compared to pregnant controls.

Next, whether differential protein expression in the plasma EV pool observed translates to differences at the mRNA level was assessed. A much more heterogeneous syncytin-1 mRNA expression was observed compared to the protein level (FIG. 3C), and differential syncytin-1 protein expression could not be extrapolated to the mRNA level. Taken together, these data demonstrated that syncytin-1 protein expression is upregulated in the plasma EV pool during pregnancy, and preeclampsia leads to changes in its expression at the protein level, but possibly not at the mRNA level. Given the specificity of syncytin-1 expression in placental trophoblasts, these data suggest that preeclampsia is associated with decreased production or increased clearance of EVs released by syncytiotrophoblasts into maternal plasma.

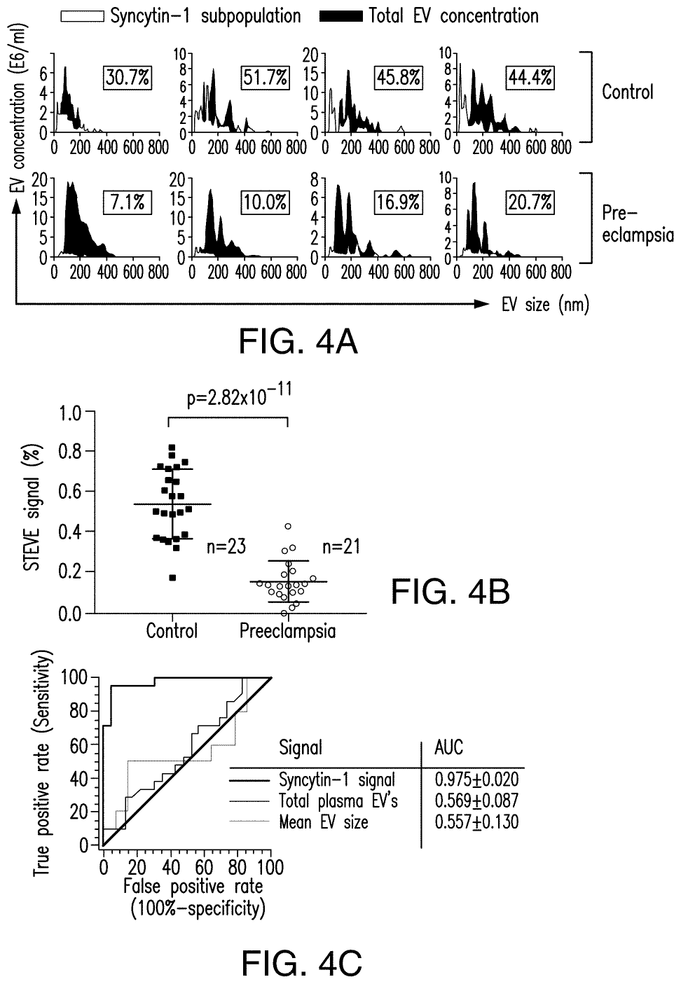

Quantitative analysis of syncytiotrophoblast EVs. Although total EV levels were indifferent between groups, Western blot analyses suggested that decreased levels of syncytin-1-expressing syncytiotrophoblast EVs are seen with preeclampsia. Therefore, the amount of syncytiotrophoblast EVs were quantified on the nanoparticle detector using anti-syncytin-1 antibody conjugated quantum dots. This demonstrated that preeclampsia subjects had lower levels of syncytin-1 expressing EVs compared to control subjects (FIG. 4A). FIG. 4A demonstrates that preeclampsia subjects had lower levels of syncytin-1 expressing EVs compared to control subjects. NanoSight panels show syncytin-1 positive EV subpopulation (empty) in relation to the total plasma EV pool (full). Syncytin-1 EV quantitative signal was significantly lower in the preeclampsia group (FIG. 4B) in accordance to the Western blot findings. Given this difference, a receiver operating characteristic curve was generated to understand the diagnostic potential of sycytin-1 EV signal to distinguish between preeclampsia versus normal pregnancy. This demonstrated an area under the curve of 0.975±0.020. Syncytin-1 EV signal threshold level of <0.316 predicted preeclampsia in this cohort with 95.2% sensitivity and 95.6% specificity (FIG. 4C). However, total plasma EV numbers and mean plasma EV size had lower accuracy profiles to predict preeclampsia (FIG. 4C). FIG. 4C demonstrates an area under the curve of 0.975±0.020 for STEV quantitative profiling, and a syncytin-1 EV signal threshold level of <0.316 predicted preeclampsia in this cohort with 95.2% sensitivity and 95.6% specificity. Total plasma EV numbers and mean plasma EV size had low diagnostic accuracy compared to STEV signal quantitation.Collectively, this data suggests that quantitative syncytiotrophoblast EV profiling, but not whole plasma EV profiling, might serve as a noninvasive diagnostic in conditions of placental pathology such as preeclampsia.

### Discussion:

Extracellular microvesicles (EVs) comprise a wide range of nanovesicles and microvesicles released by many cell types into bodily fluids including peripheral blood, urine, cerebrospinal fluid, and gastrointestinal secretions (Julich et al., 2014). These include exosomes, plasma membrane blebs, microvesicles, and apoptotic bodies. Types of EVs are typically classified by their size and mechanism of origin. Exosomes are tissue specific nanovesicles in the range of 50 to 200 nm that are derived from the multivesicular body of the cell and released into bodily fluid or extracellular space by its fusion with the cell membrane. Exosomes carry canonical and cell-specific protein and nucleic acid cargoes, and they play an important role in intercellular communication at the paracrine and systemic levels. Circulating exosome quantitative and cargo profiles are dynamic and may reflect condition-specific changes mediated upon their tissue of origin.

The findings of the present disclosure support the growing body of literature that pregnancy renders an altered EV profile status in the maternal plasma and that the fetal-derived syncytiotrophoblasts release EVs into the maternal plasma. Furthermore, conditions associated with placental pathology such as preeclampsia lead to differences in total EV quantities in maternal plasma, and more importantly significant changes in syncytiotrophoblast specific EVs may be observed. In the two cohorts, a trend towards increasing total EV quantities in maternal plasma with preeclampsia was noted. A recent study by Salomon et al. (Salomon et al., 2017) showed that increasing EV quantities are noted during latter stages of gestation, and furthermore, significantly higher number of EVs were seen in preeclampsia subjects at each gestational stage (early: 11-14 weeks, mid: 22-28 weeks, late: 32-38 weeks). Analysis of plasma EV protein content for PLAP by enzyme linked immunoabsorbent assay showed that significantly higher PLAP content was seen in preeclampsia subjects overall. In gestation matched samples, however, when the PLAP content was normalized to total plasma EV quantity, the difference in PLAP content between preeclampsia and control subjects was diminished. For early gestational stage samples, the positive and negative predictive values by receiver operating characteristic curve for exosomal PLAP content was 75% and 76% respectively in the present disclosure.

In another study analyzing third trimester maternal plasma EV samples comparing preeclampsia subjects to normal controls, total plasma EV quantities were significantly increased in preeclampsia subjects (Pillay et al., 2016), with much higher fold differences in total plasma EV concentrations than that reported by Salomon et al (Salomon et al., 2017). In early onset preeclampsia subjects compared to normotensive subjects <33-week gestation, 14.3-fold difference was seen in total plasma EV concentrations. A big difference in total plasma EV numbers was not observed between normal and preeclampsia subjects, although this analysis was performed in plasma samples from pregnant females in 2^{nd} or 3^{rd} trimester of gestation. These findings are more in accordance with Salomon et al. (Salomon et al., 2017), where there was <2-fold difference in total EV numbers between the two cohorts, with higher numbers seen in preeclampsia subjects. Pillay et al. (Pillay et al., 2016) also studied PLAP protein content in plasma EV fraction, and found increasing total PLAP content with early onset preeclampsia but not late onset preeclampsia. But when normalized to plasma EV quantities, the PLAP content to EV quantity ratio showed significantly decreased values in late onset preeclampsia subjects compared to normotensive controls. Both these studies utilized plasma PLAP protein content as a marker of placental EVs and did not directly measure PLAP surface expression on EVs. These studies suggest that preeclampsia is associated with a state of increased total EV quantities in maternal plasma but not necessarily increased syncytiotrophoblast EVs. Also, increased circulating quantity of placental markers such as PLAP might not necessarily translate to increased syncytiotrophoblast specific EV concentration in maternal plasma.

In support of the above idea, Tannetta et al. (Tannetta et al., 2013) studied microvesicles released by perfused placentas in an in vitro closed-circuit system obtained by cesarean section from normal versus preeclampsia subjects. Unlike other studies that analyzed PLAP protein content by enzyme linked immunoabsorbent assay as a surrogate for placenta specific EVs, the present disclosure measured surface expression of PLAP on microvesicles. In flow cytometry analysis of microvesicles in the 300 nm to 1 μm range, they noted decreased percentage of PLAP positive microvesicles in perfused placentas from preeclampsia subjects compared to normal controls. In addition, mean fluorescence intensity for PLAP by flow cytometry was also decreased in microvesicles from preeclampsia subjects, suggesting that PLAP expressing EV quantity and the amount of PLAP carried on each microvesicle surface was decreased in microvesicles released by placentas from preeclampsia subjects. Accordingly, densitometry analysis of microvesicle protein content by Western blot for PLAP was significantly decreased in preeclampsia samples compared to normal controls. Furthermore, size distribution analysis of microvesicles by nanoparticle detector analysis, which enables analysis of particles in 10 nm to 1000 nm range, showed that placentas perfused from preeclampsia subjects contained fewer microvesicles in the exosome size range compared to placentas from normal subjects. Taken together, these results suggest that perfused placentas from preeclampsia subjects release decreased PLAP positive microvesicles, with lower PLAP surface expression per microvesicle and decreased microvesicles in the exosome range. Therefore, placental pathology associated with preeclampsia might manifest as a decrease in EV production with decreased expression of placenta specific marker on syncytiotrophoblast microvesicles, especially exosomes. The results support these findings observed in a placenta perfusion model. Even though a general trend towards higher numbers of EVs in preeclampsia subjects was found, there was a significant reduction in syncytiotrophoblast specific EV signal in preeclampsia maternal plasma samples. This decrease in syncytiotrophoblast specific EV signal can reflect a lower number of placenta specific EVs, lower amounts of placenta specific marker on EVs, or a combination of both. These findings are in accordance to the view that tissue injury leads to decreased production of tissue specific exosomes.

In addition to PLAP, which is the most studied protein as a placenta specific marker, syncytin-1 and syncytin-2 proteins have also been reported to be altered in placental tissues from preeclampsia subjects (Knerr et al., 2002, Vargas et al., 2011). Syncytin-1 and -2 are human endogenous retrovirus envelope proteins shown to play a critical role in trophoblast fusion, a process vital to formation of multinucleated syncytiotrophoblasts. Reduced expression of both syncytin-1 and syncytin-2 mRNA was shown in primary trophoblasts cultured from placentas of mothers with preeclampsia compared to normal controls. By Western blot analysis, decreased protein levels of syncytin-1 were noted in trophoblasts from subjects with severe preeclampsia, but a more pronounced difference in protein expression was noted with syncytin-2. The same group also analyzed EVs from maternal serum isolated using a polymer-based EV isolation kit. Densitometry analysis of serum EV protein content by Western blot showed no significant differences in syncytin-1 levels between control and preeclampsia samples, but syncytin-2 levels were significantly decreased in preeclampsia samples.

It is important to note that decreased syncytin-1 EV signal in preeclampsia subjects detected by nanoparticle detector analysis may not necessarily mean that there is decreased production of EVs by syncytiotrophoblasts under this condition. It could very well be that in preeclampsia, the density of syncytin-1 surface expression on EVs is reduced, which could result in decreased synctin-1 EV signal detection without a decrease in overall EV production by syncytiotrophoblasts. Accordingly, just because increased EV quantities in maternal plasma from preeclampsia subjects were found does not mean that these increased numbers are due to increased EV production by placental syncytiotrophoblasts. Certain literature support that overall a higher number of EVs are detected in maternal plasma from preeclampsia subjects compared to normal pregnancy. Also, there is evidence that increasing numbers of EVs are seen in maternal plasma during early to late gestation. Therefore, interpretation of the diagnostic potential of syncytiotrophoblast EVs must be investigated taking into consideration changes in peripheral blood EV profiles in a gestation-specific and condition-specific manner. To this effect, it will be important to understand gestation-specific changes in STEVs during normal pregnancy, before understanding specific changes associated with preeclampsia. Lastly, certain literature on EVs in the obstetrics diagnostics space reflects studies utilizing different EV isolation techniques, and various methods of signal quantitation. This Example shows that plasma EVs can be used to diagnose placental pathologies, such as preeclampsia. Further, to carefully understand the potential of plasma EVs profiling for noninvasive diagnosis of placental pathologies, it can be important to standardize these methodologies and quantitation assays so that internal and intergroup variability is reduced.

### REFERENCES

Tranquilli A L, Dekker G, Magee L, et al. The classification, diagnosis and management of the hypertensive disorders of pregnancy: A revised statement from the ISSHP. Pregnancy Hypertens 2014; 4:97-104.

Tranquilli A L, Brown M A, Zeeman G G, Dekker G, Sibai B M. The definition of severe and early-onset preeclampsia. Statements from the International Society for the Study of Hypertension in Pregnancy (ISSHP). Pregnancy Hypertens 2013; 3:44-7.

Magee L A, Pels A, Helewa M, Rey E, von Dadelszen P, Committee SHG. Diagnosis, evaluation, and management of the hypertensive disorders of pregnancy: executive summary. J Obstet Gynaecol Can 2014; 36:575-76.

Wallis A B, Saftlas A F, Hsia J, Atrash H K. Secular trends in the rates of preeclampsia, eclampsia, and gestational hypertension, United States, 1987-2004. Am J Hypertens 2008; 21:521-6.

Report of the National High Blood Pressure Education Program Working Group on High Blood Pressure in Pregnancy. Am J Obstet Gynecol 2000; 183:S1-S22.

Lindheimer M D, Taler S J, Cunningham F G. Hypertension in pregnancy. J Am Soc Hypertens 2010; 4:68-78.

Bulletins—Obstetrics ACoP. ACOG practice bulletin. Diagnosis and management of preeclampsia and eclampsia. Number 33, January 2002. Obstet Gynecol 2002; 99:159-67.

Fukui A, Yokota M, Funamizu A, et al. Changes of NK cells in preeclampsia. Am J Reprod Immunol 2012; 67:278-86.

Nelissen E C, van Montfoort A P, Dumoulin J C, Evers J L. Epigenetics and the placenta. Hum Reprod Update 2011; 17:397-417.

Pijnenborg R, Vercruysse L, Hanssens M. Fetal-maternal conflict, trophoblast invasion, preeclampsia, and the red queen. Hypertens Pregnancy 2008; 27:183-96.

George E M, Granger J P. Recent insights into the pathophysiology of preeclampsia. Expert Rev Obstet Gynecol 2010; 5:557-66.

Roberts J M, Gammill H S. Preeclampsia: recent insights. Hypertension 2005; 46:1243-9.

LaMarca B D, Gilbert J, Granger J P. Recent progress toward the understanding of the pathophysiology of hypertension during preeclampsia. Hypertension 2008; 51:982-8.

Brosens I A, Robertson W B, Dixon H G. The role of the spiral arteries in the pathogenesis of preeclampsia. Obstet Gynecol Annu 1972; 1:177-91.

Damsky C H, Fisher S J. Trophoblast pseudo-vasculogenesis: faking it with endothelial adhesion receptors. Curr Opin Cell Biol 1998; 10:660-6.

Wang A, Rana S, Karumanchi S A. Preeclampsia: the role of angiogenic factors in its pathogenesis. Physiology (Bethesda) 2009; 24:147-58.

Mutter W P, Karumanchi S A. Molecular mechanisms of preeclampsia. Microvasc Res 2008; 75:1-8.

Thadhani R, Kisner T, Hagmann H, et al. Pilot study of extracorporeal removal of soluble fms-like tyrosine kinase 1 in preeclampsia. Circulation 2011; 124:940-50.

Mitchell M D, Peiris H N, Kobayashi M, et al. Placental EMVs in normal and complicated pregnancy. Am J Obstet Gynecol 2015; 213:S173-81.

Germain S J, Sacks G P, Sooranna S R, Sargent I L, Redman C W. Systemic inflammatory priming in normal pregnancy and preeclampsia: the role of circulating syncytiotrophoblast microparticles. J Immunol 2007; 178:5949-56.

Sarker S, Scholz-Romero K, Perez A, et al. Placenta-derived EMVs continuously increase in maternal circulation over the first trimester of pregnancy. J Transl Med 2014; 12:204.

Tannetta D, Collett G, Vatish M, Redman C, Sargent I. Syncytiotrophoblast extracellular vesicles—Circulating biopsies reflecting placental health. Placenta 2017; 52:134-38.

Tannetta D, Masliukaite I, Vatish M, Redman C, Sargent I. Update of syncytiotrophoblast derived extracellular vesicles in normal pregnancy and preeclampsia. J Reprod Immunol 2017; 119:98-106.

Vallabhajosyula P, Korutla L, Habertheuer A, et al. Tissue-specific EMV biomarkers for noninvasively monitoring immunologic rejection of transplanted tissue. J Clin Invest 2017; 127:1375-91.

Lotvall J, Hill A F, Hochberg F, et al. Minimal experimental requirements for definition of extracellular vesicles and their functions: a position statement from the International Society for Extracellular Vesicles. J Extracell Vesicles 2014; 3:26913.

Valadi H, Ekstrom K, Bossios A, Sjostrand M, Lee J J, Lotvall J O. Exosome-mediated transfer of mRNAs and microRNAs is a novel mechanism of genetic exchange between cells. Nat Cell Biol 2007; 9:654-9.

Cuffe J S M, Holland O, Salomon C, Rice G E, Perkins A V. Review: Placental derived biomarkers of pregnancy disorders. Placenta 2017; 54:104-10.

Pillay P, Maharaj N, Moodley J, Mackraj I. Placental EMVs and pre-eclampsia: Maternal circulating levels in normal pregnancies and, early and late onset pre-eclamptic pregnancies. Placenta 2016; 46:18-25.

Salomon C, Guanzon D, Scholz-Romero K, et al. Placental EMVs as early biomarker of preeclampsia—Potential role of exosomal microRNAs across gestation. J Clin Endocrinol Metab 2017.

Salomon C, Rice G E. Role of Exosomes in Placental Homeostasis and Pregnancy Disorders. Prog Mol Biol Transl Sci 2017; 145:163-79.

Tan K H, Tan S S, Sze S K, Lee W K, Ng M J, Lim S K. Plasma biomarker discovery in preeclampsia using a novel differential isolation technology for circulating extracellular vesicles. Am J Obstet Gynecol 2014; 211:380 el-13.

Vargas A, Moreau J, Landry S, et al. Syncytin-2 plays an important role in the fusion of human trophoblast cells. J Mol Biol 2009; 392:301-18.

Vargas A, Toufaily C, LeBellego F, Rassart E, Lafond J, Barbeau B. Reduced expression of both syncytin 1 and syncytin 2 correlates with severity of preeclampsia. Reprod Sci 2011; 18:1085-91.

Vargas A, Zhou S, Ethier-Chiasson M, et al. Syncytin proteins incorporated in placenta EMVs are important for cell uptake and show variation in abundance in serum EMVs from patients with preeclampsia. FASEB J 2014; 28:3703-19.

Habertheuer A, Korutla L, Rostami S, et al. Donor tissue-specific EMV profiling enables noninvasive monitoring of acute rejection in mouse allogeneic heart transplantation. J Thorac Cardiovasc Surg 2018.

Habertheuer A, Korutla L, Rostami S, et al. Donor lung specific EMV profiles for noninvasive monitoring of acute rejection in a rat orthotopic lung transplant model. J Heart Lung Transplant 2018.

American College of O, Gynecologists, Task Force on Hypertension in P. Hypertension in pregnancy. Report of the American College of Obstetricians and Gynecologists' Task Force on Hypertension in Pregnancy. Obstet Gynecol 2013; 122:1122-31.

DeLong E R, DeLong D M, Clarke-Pearson D L. Comparing the areas under two or more correlated receiver operating characteristic curves: a nonparametric approach. Biometrics 1988; 44:837-45.

Sung K U, Roh J A, Eoh K J, Kim E H. Maternal serum placental growth factor and pregnancy-associated plasma protein A measured in the first trimester as parameters of subsequent pre-eclampsia and small-for-gestational-age infants: A prospective observational study. Obstet Gynecol Sci 2017; 60:154-62.

Poon L C, Maiz N, Valencia C, Plasencia W, Nicolaides K H. First-trimester maternal serum pregnancy-associated plasma protein-A and pre-eclampsia. Ultrasound Obstet Gynecol 2009; 33:23-33.

Kalousova M, Murayska A, Zima T. Pregnancy-associated plasma protein A (PAPP-A) and preeclampsia. Adv Clin Chem 2014; 63:169-209.

Bersinger N A, Smarason A K, Muttukrishna S, Groome N P, Redman C W. Women with preeclampsia have increased serum levels of pregnancy-associated plasma protein A (PAPP-A), inhibin A, activin A and soluble E-selectin. Hypertens Pregnancy 2003; 22:45-55.

Cozzi V, Garlanda C, Nebuloni M, et al. PTX3 as a potential endothelial dysfunction biomarker for severity of preeclampsia and IUGR. Placenta 2012; 33:1039-44.

Carty D M, Delles C, Dominiczak A F. Novel biomarkers for predicting preeclampsia. Trends Cardiovasc Med 2008; 18:186-94.

Ree P H, Hahn W B, Chang S W, et al. Early detection of preeclampsia using inhibin a and other second-trimester serum markers. Fetal Diagn Ther 2011; 29:280-6.

Teixeira P G, Cabral A C, Andrade S P, et al. Placental growth factor (P1GF) is a surrogate marker in preeclamptic hypertension. Hypertens Pregnancy 2008; 27:65-73.

Keshavarzi F, Mohammadpour-Gharehbagh A, Shahrakipour M, et al. The placental vascular endothelial growth factor polymorphisms and preeclampsia/preeclampsia severity. Clin Exp Hypertens 2017; 39:606-11.

Tache V, LaCoursiere D Y, Saleemuddin A, Parast M M. Placental expression of vascular endothelial growth factor receptor-1/soluble vascular endothelial growth factor receptor-1 correlates with severity of clinical preeclampsia and villous hypermaturity. Hum Pathol 2011; 42:1283-8.

Fu G, Ye G, Nadeem L, et al. MicroRNA-376c impairs transforming growth factor-beta and nodal signaling to promote trophoblast cell proliferation and invasion. Hypertension 2013; 61:864-72.

Jiang L, Long A, Tan L, et al. Elevated microRNA-520 g in pre-eclampsia inhibits migration and invasion of trophoblasts. Placenta 2017; 51:70-75.

Julich H, Willms A, Lukacs-Kornek V, Kornek M. Extracellular vesicle profiling and their use as potential disease specific biomarker. Front Immunol 2014; 5:413.

Tannetta D S, Dragovic R A, Gardiner C, Redman C W, Sargent I L. Characterisation of syncytiotrophoblast vesicles in normal pregnancy and pre-eclampsia: expression of Flt-1 and endoglin. PLoS One 2013; 8:e56754.

Knerr I, Beinder E, Rascher W. Syncytin, a novel human endogenous retroviral gene in human placenta: evidence for its dysregulation in preeclampsia and HELLP syndrome. Am J Obstet Gynecol 2002; 186:210-3.

Various publications, patents and patent applications are cited herein, the contents of which are hereby incorporated by reference in their entireties.

What is claimed is:

1. A method for diagnosing a subject with preeclampsia, comprising:
(a) detecting and/or isolating one or more biomarkers from a biological sample; and
(b) diagnosing preeclampsia in the subject when there is a change in the presence and/or level of the one or more biomarkers.

2. A method for diagnosing a subject with preeclampsia, comprising:
(a) isolating, purifying, and/or identifying one or more syncytiotrophoblast-derived microvesicles from a biological sample;
(b) analyzing one or more biomarkers associated with the syncytiotrophoblast-derived microvesicles; and
(c) diagnosing the subject with preeclampsia when there is a change in the presence and/or level of the one or more biomarkers as compared to a reference control level.

3. The method of claim 1, wherein the biomarker is a protein and/or a nucleic acid.

4. The method of claim 2, wherein the biomarker is a protein and/or nucleic acid.

5. The method of any one of claims 1-4, wherein the biomarker is syncytin-1 and wherein the reduction in the level of syncytin-1 compared to the reference control is indicative that the subject has preeclampsia.

6. A method for diagnosing a subject with preeclampsia, comprising:
(a) isolating, purifying, and/or identifying one or more syncytiotrophoblast-derived microvesicles from the biological sample;
(b) analyzing the number of syncytiotrophoblast-derived microvesicles expressing syncytin-1; and
(c) diagnosing the subject with preeclampsia when the number of syncytin-l-expressing syncytiotrophoblast-derived microvesicles is reduced compared to a reference control.

7. The method of claim 6, further comprising detecting a marker specific for syncytiotrophoblasts and/or microvesicles.

8. The method of claim 7, wherein the marker specific for syncytiotrophoblasts is a protein.

9. The method of claim 8, wherein the protein is a surface protein.

10. The method of any one of claims 7-9, wherein the marker specific for syncytiotrophoblasts is selected from the group consisting of syncytin-1, PLAP, and a combination thereof.

11. The method of claim 7-10, wherein the marker specific for microvesicles is selected from the group consisting of flotillin-1, CD63, CD9, CD81, TSG101, and a combination thereof.

12. A method for treating a subject with preeclampsia, comprising:
(a) isolating, purifying, and/or identifying one or more syncytiotrophoblast-derived microvesicles from a biological sample;
(b) analyzing one or more biomarkers associated with the syncytiotrophoblast-derived microvesicles;
(c) diagnosing the subject with preeclampsia when there is a change in the presence and/or level of the one or more biomarkers as compared to a reference control level; and
(d) treating the subject diagnosed with preeclampsia.

13. The method of claim 12, wherein treating the subject diagnosed with preeclampsia comprises one or more of the following:
(a) administration of an anti-hypertensive medication;
(b) delivery;
(c) administration of a corticosteroid;
(d) bed rest; and/or
(e) administration of an anti-convulsant medication.

14. The method of any one of claims 1-13, wherein the reference control is the level in a pregnant subject that does not have preeclampsia.

15. A method for preparing a syncytiotrophoblast-derived microvesicles fraction from a pregnant subject, comprising:
(a) extracting one or more microvesicles from a biological sample of a pregnant subject;
(b) producing a fraction of the microvesicles extracted in (a) by selectively enriching syncytiotrophoblast-derived microvesicles;
and (c) measuring one or more biomarkers.

16. The method of claim 15, wherein the enriching comprises contacting the sample with an agent binding a cell-specific marker.

17. The method of claim 15, wherein the cell-specific marker is selected from the group consisting of syncytin-1, placental alkaline phosphatase (PLAP), plac-1, flotillin-1, CD63, CD9, CD81, and TSG101.

18. The method of claim 15 or 17, wherein the cell-specific marker is syncytin-1.

19. The method of any one of claims 15-18, further comprising size discrimination of microvesicles.

20. A method for isolating and/or purifying syncytiotrophoblast-derived microvesicles, comprising:
(a) obtaining a biological sample from a subject;
(b) isolating and/or purifying one or more microvesicles from the biological sample; and
(c) isolating, purifying, and/or identifying one or more syncytiotrophoblast-derived microvesicles from the one or more microvesicles of (b) by detecting a marker specific for syncytiotrophoblasts.

21. The method of claim 20, wherein the marker specific for syncytiotrophoblasts is a protein.

22. The method of claim 21, wherein the protein is a surface protein.

23. The method of any one of claims 20-22, wherein the marker specific for syncytiotrophoblasts is selected from the group consisting of syncytin-1, PLAP, and a combination thereof.

24. The method of any one of claims 20-23, wherein isolating and/or purifying one or more microvesicles from the biological sample comprises detecting a marker specific for microvesicles.

25. The method of claim 24, wherein the marker specific for microvesicles is selected from the group consisting of flotillin-1, CD63, CD9, CD81, TSG101, and a combination thereof.

26. The method of any one of claims 1-25, wherein the subject is human.

27. The method of any one of claims 1-26, wherein the biological sample is a blood sample.

28. A kit for diagnosing and/or monitoring a subject with preeclampsia, comprising reagents useful for detecting a marker specific to a syncytiotrophoblast-derived microvesicle.

29. The kit of claim 28, comprising a packaged probe and primer set, arrays/microarrays, marker-specific antibodies or marker-specific antibody-conjugated beads or quantum dots.

30. The kit of claim 28, comprising a pair of oligonucleotide primers, suitable for polymerase chain reaction or nucleic acid sequencing, for detecting the marker.

31. The kit of claim 28, comprising a monoclonal antibody or antigen-binding fragment thereof, or a polyclonal antibody or antigen-binding fragment thereof, for detecting the marker.

32. The kit of any one of claims 28-32, wherein the marker specific to a syncytiotrophoblast-derived microvesicle is selected from the group consisting of syncytin-1, PLAP, and a combination thereof.
